(19) United States
(12) Patent Application Publication
(10) Pub. No.: US 2005/0025819 A1
(43) Pub. Date: Feb. 3, 2005

(54) Materials and methods for making improved micelle compositions

(76) Inventors: Hayat Onyuksel, Western Springs, IL (US); Israel Rubinstein, Highland Park, IL (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/496,819

(22) PCT Filed: Nov. 27, 2002
(86) PCT No.: PCT/US02/38186

Related U.S. Application Data
(63) Continuation-in-part of application No. 09/995,403, filed on Nov. 27, 2001.
Application No. 09/995,403 is a continuation-in-part of application No. 09/239,069, filed on Jan. 27, 1999, now Pat. No. 6,217,886.
Application No. 09/995,403 is a continuation-in-part of application No. 09/462,819, filed on May 18, 2000, now Pat. No. 6,322,810.
(60) Provisional application No. 60/052,078, filed on Jul. 14, 1997.

(51) Int. Cl. A61K 9/127

(52) U.S. Cl. 424/450; 424/78.27

(57) ABSTRACT

Provided are methods of treatment of many different diseases and disorders using micelle and sterically stabilized crystalline compounds of the invention.

### BACKGROUND OF THE INVENTION

The present invention relates generally to biologically activecompounds and more specifically to compounds, peptides, proteins, fragments, analogs, and modulators thereof which are amphipathic, i.e., have both hydrophilic and hydrophobic portions. Specifically, the invention relates to improved methods for the delivery and presentation of amphipathic peptides, proteins, fragments, analogs, and modulators thereof alone or conjugated to other compounds in association with micelles or monomers of micelles diagnostic, therapeutic, cosmetic and organ tissue and cell preservative uses. The invention also provides methods for the delivery of compounds that are insoluble or nearly insoluble in an aqueous solution. Specifically, the invention provide methods to produce sterically stabilized crystalline products comprised of a crystallized insoluble compound coated with a lipid surface.

Of particular interest to the present invention are the biologically active amphipathic peptides which are members of the family of peptide compounds including, but not limited to, vasoactive intestinal peptide (VIP), growth hormone releasing factor (GRF), hypocretins, peptide histidine isoleucine (PHI), peptide histidine methionine (PHM), pituitary adenylate cyclase activating peptide (PACAP), gastric inhibitory hormone (GIP), hemodermin, the growth hormone releasing hormone (GHRH), sauvagine and urotensin I, secretin, glucagon, galanin, endothelin, calcitonin, α₁-proteinase inhibitor, angiotensin II, corticotropin releasing factor, antibacterial peptides and proteins in general, surfactant peptides and proteins, α-MSH, adrenolmedullin, ANF, IGF-1, α2 amylin, orphanin, and orexin. More specifically, the invention relates to improved therapeutic methods for delivering peptides in the VIP/GRF or IL-2 family of peptides, as well as other amphipathic peptides, to targeted tissues through use of improved micelle compositions comprising a member of the VIP/GRF or IL-2 family of peptides, amphipathic peptides in general, proteins, and biologically active analogues, fragments and modulators thereof.

VIP is a 28-amino acid neuropeptide which is known to display a broad profile of biological actions and to activate multiple signal transducing pathways. See, Said, Peptides 5 (Suppl. 1):149-150 (1984) and Paul and Ebadi, Neurochem. Int. 23:197-214 (1993). A Schiff-Edmundson projection of VIP as a π-helix reveals segregation of apolar and polar residues onto the opposite faces of the helix and that this amphipathic character is also evident when VIP is modeled as a distorted α-helix, which is reported in Musso, et al., Biochemistry 27:8147-8181 (1988). A correlation between the helix-forming tendency of VIP analogues and their biological activity is described in Bodanszky et al., Bioorgan. Chem: 3:133-140 (1974). In pure water, the spectral characteristics of VIP are consistent with those of a random coil. However, organic solvents and anionic lipids induce helical-information in the molecule. See, Robinson et al., Biopolymers 21:1217-1228 (1983); Hamed, et al., Biopolymers 22:1003-1021 (1983); and Bodanszky, et al., Bioorganic Chem. 3:133-140 (1974).

Short peptides capable of forming amphipathic helices are known to bind and penetrate lipid bilayers. See, Kaiser and Kezdy, Ann. Rev. Biophys. Biophysical Chem. 15:561-581 (1987) and Sansom, Prog. Biophys. Molec. Biol. 55:139-235 (1991). Examples include model peptides like (LKKLLKL-), which are disclosed in DeGrado and Lear, J. Am. Chem. Soc. 107:7684-7689 (1985), and the 26-residue bee venom peptide, melittin, disclosed in Watata and Gwozdzinski, Chem-Biol. Interactions 82:135-149 (1992). Possible mechanisms for the binding include alignment of peptide monomers parallel to the surface of the bilayer mediated by electrostatic interactions between polar amino acids and phospholipid head groups, and insertion of peptide aggregates into the apolar bilayer core, stabilized in part, by the hydrophobic effect. See, Sansom, Prog. Biophys. Molec. Biol. 55:139-235 (1991).

VIP belongs to a family of homologous peptides, other members of which include peptide histidine isoleucine (PHI), peptide histidine methionine (PHM), growth hormone releasing factor (GRF), hypocretins, pituitary adenylate cyclase activating peptide (PACAP), gastric inhibitory hormone (GIP), hemodermin, the growth hormone releasing hormone (GHRH), sauvagine and utotensin I, secretin and glucagon. Like VIP, the other members of the VIP/GRF family of peptides, and biologically active analogues thereof, can form amphipathic helices capable of binding lipid bilayers. The biological action of members of the VIP/GRF family of peptides are believed to be mediated by protein receptors expressed on the cell surface and intracellular receptors and it has recently been demonstrated that calmodulin is likely to be the intracellular receptor for VIP [Stallwood, et al., J. Bio. Chem. 267:19617-19621 (1992); and Stallwood, et al., FASEB J. 7:1054 (1993)].

Bodanszky et al., Bioorgan. Chem. 3:133-140 (1974) were the first to study the conformation of VIP through optical rotary dispersion and circular dichroism spectrum. They showed structural differences in VIP, depending on the hydrophobicity of the solvent in which VIP was dissolved. The VIP-in-water spectrum revealed a mostly random coil structure (about 80%). However, addition of organic solvents, such as trifluoroethanol (TFE) or methanol, even at low concentration induced a pronounced shift to a helical structure. The authors suggested that these effects of the organic solvents on the structure of the peptide would coincide with receptor conditions, and therefore, the helical conformation of VIP would correspond to an “active architecture” required for its biological activity. These early studies were in agreement with the findings of Robinson et al., Biopolymers 21:1217-1228 (1982), who analyzed the conformation of VIP, and two of its family members, secretin and glucagon, in water, anionic detergents, and anionic lipids (PA and phosphatidylglycerol (PG)). They showed an increase in the helix formation probability by arginyl, histidyl, and lysil residues, corresponding in all three peptides to their 13-20 amino acid region. A predominantly disordered structure was again observed for VIP in aqueous solvents, and zwitterionic lipids, suggesting that the charge of the polar head group plays an important role in helix formation. Using circular dichroism (CD) spectra studies with 40% HFIP/H₂O mixture and ¹H-NMR studies Fournier et al., Peptides 5:160-177 (1984), showed that the 15-28 portion of the VIP segment forms an α-helix in the presence of organic solvent. A complete structural study of the native VIP in 40% TFE was performed by Theriault et al., Biopolymers 31:459-464 (1991) using two-dimensional ¹H-NMR spectroscopy. Their results were similar to the ones obtained by Fry et al., Biochemistry 28:2399-2409 (1989) who investigated VIP in 25% methanol/water. They described two helical segments between the amino acids 7-15 and 19-27 linked by a random coil peptide chain portion that granted mobility to the molecule.

Finally several groups worked on the development of more potent analogs of VIP as potential therapeutic agents, since the native peptide had a very low bioavailability. Interestingly, all of them modified the sequence of VIP to enhance its helicity and amphiphilicity. VIP structure-activity relationship were studied extensively by Bolin and his collaborators (Fry et al., Biochemistry 28:2399-2409 (1989); Bolin et al., Biopolymers 37:57-66 (1995). Among their results, the enhancement of the helical structure by specific substitutions of amino acid residues was proportionally related to an increase in potency, and the pharmacoactive functional group of the VIP was found to consist of multiple binding sites throughout the entire peptide sequence. Helix based analogs of VIP were also developed by Musso et al., Biochemistry 27:8174-8181 (1988) that showed greater interactions with receptors. Stearyl-Norleucine-VIP analog that has a 100-fold greater potency was designed by Gozes et al., Endocrinology 134:2121-2125 (1994), for noninvasive impotence treatment and neurodegenerative diseases Gozes et al. J. Pharmacol. Exp. Ther. 273:161-167 (1996). The addition of fatty acid moiety and the amino acid substitutions increased the lipophilicity of the peptide, which was believed to improve biological membrane penetration.

In summary, VIP and other members of the superfamily have been shown to adopt a helical conformation in hydrophobic environments, provided by organic solvent, and the helical structure of the VIP increases with an increase in the hydrophobicity of the environment. This helical motif found in the central part of the peptide, which is rich in basic, hydrophobic residues, forms an amphiphilic structure that may facilitate the binding to receptors and promote direct interactions with membrane lipids, causing an increase in bioactivity. Furthermore, it is possible that the helical structure of VIP also contributes to an increased stability, by protecting specific sites particularly sensitive to proteolytic degradation.

As reviewed by Gozes et al., Mol. Neurobiol. 3:201-236 (1989), immunofluorescence and radioimmunoassay techniques demonstrated the wide but selective distribution of VIP in the central and peripheral nervous systems. In the brain, the highest density of VIP-rich neurons occur in the hypothalamus, particularly in the suprachiasmatic and paraventricular nuclei and in the cerebral cortex. VIP concentrations are higher in the hypophyseal portal blood than in the peripheral blood, indicating secretion of the peptide by the hypothalamus and its transport to the adenohypophysis. In the peripheral nervous system, VIP-immunoreactive nerves are found in fibers and terminals that supply blood vessels, nonvascular smooth muscle, and glandular acini and ducts in many organs. Coexistence of VIP with acetylcholine in cholinergic neurons is also well-documented. Some VIP nerves have recently been acknowledged to be components of the autonomic nervous system. Furthermore, Muller et al., Mol. Neurobiol. 10:115-134 (1995) showed that a distinct groups of cells, such as platelets, mast cells, skin cells, neutrophils, and retinal amacrine cells appear to be able to synthesize and release VIP.

The physiologic effects of VIP are largely mediated by its binding to specific cell receptors. Hirata et al., Biochem. Biophys. Res. Comm. 132:1079-1087 (1985) described two specific receptor binding sites for VIP, one low-, one high-affinity, on cultured vascular smooth muscle cells from rat aorta, that were distinct from β-adrenergic receptors. From a molecular aspect two distinct polyvalent VIP receptors were distinguished after cloning of cDNAs. The first, VIP₁, receptor is similar to the secretin receptor also called PACAP type II receptor, is expressed in intestine, lung, liver, muscle cells, ovaries, and various brain regions (Sreedharan et al., Biochem. Biophys. Res. Comm. 203:141-148 (1994)). The second, VIP₂ receptor is closer to the GRF binding site and has a distinct distribution in the central nervous system (Lutz et al., FEBS Let. 334:3-8 (1993)). Recent studies have also indicated that VIP action can be non-receptor mediated (Séjourné et al., Am. J. Physiol. 273:R287-R292 (1997)).

Although studied for many years, most of the intracellular signaling cascades of VIP remain to be elucidated. Most common cellular action observed in many cells is the increased production of intracellular cyclic adenosine monophosphate (cAMP), via the stimulation of adenylate cyclase. The subsequent steps of cAMP-induced pathways are still highly speculative. Conversely, several observations indicate the existence of cAMP-independent signal transduction cascades. Sreedharan et al., Biochem. Biophys. Res. Comm. 203:141-148 (1994) recently found that VIP₁ receptor induced two separate pathways in one cell type, i.e. activation of adenylate cyclase and increase in intracellular Ca²⁺. Stimulation of adrenal medulla and cervical ganglion by VIP were shown to increase the generation of inositol 1,4,5 triphosphate (IP₃) and intracellular Ca²⁺ (Malhotra et al., J. Biol. Chem. 263:2123-2126 (1988)). Moreover, it has been proposed that internalized VIP could bind to nuclear receptors and activate protein kinase C (Omary et al., Science 238:1578-1580 (1987); Zorn et al., Biochem. Pharmacol. 40:2689-2694 (1990)).

The pleiotropic distribution of VIP is correlated with its involvement in a broad spectrum of biological activities, and growing evidence suggests that VIP plays a major role in regulating a variety of important functions in many organs. Physiological actions of VIP have been reported on the cardiovascular, respiratory, reproductive, digestive, immune, and central nervous systems, as well as metabolic, endocrine and neuroendocrine functions (for review, Said, Trends Endocrinol. Metab. 2:107-112 (1991)). In many cases, VIP acts as a neurotransmitter or neuromodulator and is released into the local circulation at small concentrations. Among the functions that VIP is believed to mediate or promote (Said, Trends Endocrinol. Metab. 2:107-112 (1991) Paul et al., Neurochem. Int. 23:197-214 (1993)), are vasodilation of cerebral, coronary, peripheral, and pulmonary blood vessels, linked to the regulation of vascular tone; the relaxation of gastrointestinal, uterine, and tracheobroncial smooth muscles; exocrine secretion, water and anions by intestinal, respiratory, and pancreatic epithelia; stimulation of the male and female activity and responses; release and regulation of neuroendocrine functions (renin release, melatonin secretion); inhibition of the immune system (inhibition of platelet aggregation); and stimulation and protection of neuronal cells.

New VIP functions such as inhibition of vascular smooth muscle cell growth and small-cell lung carcinoma (SCLC) cells (Said et al., Proc. Natl. Acad. Sci. U.S.A., 95(24):14373-8 (1998)), and proliferation of cultured human keratinocytes, the release of neutrophic and growth factors involved in cell differentiation and ontogeny, and antioxidant properties have been recently proposed but still need additional studies (Muller et al., Mol. Neurobiol. 10:115-134 (1995); Said, Trends Endocrinol. Metab. 2:107-112 (1991)).

Some human diseases today are known to be associated with the deficiency in the release of VIP. The deficiency of VIP has been linked to the pathogenesis of several diseases, such as cystic fibrosis, diabetic impotence, congenital mengacolon in Hirschsprung's disease, and achalasia of the esophagus. Furthermore, VIP insufficiency may be a cause of bronchial hyperactivity in asthmatic airways since VIP is known to mediate airway relaxation in humans, and lung tissues of asthmatic patients showed a selective absence of VIP nerves (Ollerenshaw et al., N. Engl. J. Med. 320:1244-1248 (1989)). Finally, Avidor et al., Brain Res. 503:304-307.(1989) observed an increase in brain VIP gene expression in a rat model for spontaneous hypertension, thought to be associated with the pathophysiology of the disease.

On the other hand, the excessive release of VIP has been linked to the pathogenesis of few diseases. One of the pathological syndromes is so-called pancreatic cholera (“VIPOMA”), a watery diarrhea-hypocholaremia-hypochloridria condition (Krejs, Ann. N.Y. Acad. Sci. 527:501-507 (1988)). Certain tumors, especially pancreatic, bronchogenic, and neurogenic, have been associated with elevated circulatory levels of VIP. In addition, it has also been suggested that increased levels of neuropeptides, including VIP, are found in neonatal blood of children with autism (Nelson, et al., American Journal of Epidemiology 151 (11 Supplement):pS3 Jun. 1,2000).

Due to the numerous physiological actions of VIP, the use of VIP as a drug has been of growing interest. The potential therapeutic developments of VIP include treatment of diseases where regional blood flow is deprived. These include hypertension by reducing systemic vascular overload, left ventricular failure, congestive heart failure, and coronary or peripheral ischemia. VIP infusion in man for 10 hours was shown to reduce total peripheral resistance by 30% and increase forearm blood flow by 270% (Frase et al., Am. J. Cardiol. 60:1356-1361 (1987)). Moreover, Smiley, Am. J. Med. Sci. 304:319-333 (1992) showed VIP-immunoreactive nerves in the skin and plasma levels of VIP were found to be low in patients with schleroderma, thus treatment with VIP may restore this impaired response. Other diseases which could be treated by administration of VIP include treatment of asthmatic bronchospasm. VIP has been shown to protect against bronchoconstriction in asthmatic patients and as a relaxant of tracheobronchial smooth muscle (Morice et al., Lancet 26 2(8361):1225-1227 (1983)). Its anti-inflammatory properties could further enhance its therapeutic value in asthma (Said, Biomed. Res. 13 (Suppl. 2):257-262 (1992)). Administration of VIP could also be used in the prevention and/or reduction of tissue injury. The peptide has been described to prevent neuronal cell death produced by the external envelope protein gp120 of the human immunodeficiency virus in vitro (Gozes et al., Mol. Neurobiol. 3:201-236 (1989); Hökfelt, Neuron. 7:867-879 (1991)), which may lead to a potential therapy for AIDS dementia as well as treatment of Alzheimer's disease. Likewise, the acute inflammatory lung injury induced by a variety of insults including oxidant stress was diminished by the presence of VIP (Berisha et al., Am. J. Physiol. 259:L151 -L155 (1990)). VIP added to certain pneumoplegic solutions was also shown to improve rat lung preservation before transplantation (Alessandrini et al., Transplantation 56:964-973 (1993)).

A major factor limiting in vivo administration of VIP has been its reduced bioavailability at target tissues mostly because of proteolytic degradation, hydrolysis, and/or a multiplicity of conformations adopted by the peptide. It has been speculated that intracellular delivery of VIP alone and/or VIP-calmodulin mixtures could bypass the requirement for cell-surface binding of the peptide and thus enhance the biological actions of the peptide. Provision of the peptides expressed in and on liposomes or micelles would possibly permit intracellular delivery, since lipid bilayers of liposomes and micelles are known to fuse with the plasma membrane of cells and deliver entrapped contents into the intracellular compartment.

Liposomes are microscopic spherical structures composed of phospholipids which were first discovered in the early 1960s (Bangham et al., J. Mol. Biol. 13:238 (1965)). In aqueous media, phospholipid molecules being amphiphilic spontaneously organize themselves in self-closed bilayers as a result of hydrophilic and hydrophobic interactions. The resulting vesicles, called liposomes, therefore encapsulate in their interior part of the aqueous medium in which they are suspended, a property that makes them potential carriers for biologically active hydrophilic molecules and drugs in vivo. Lipophilic agents could also be transported, embedded in the liposomal membrane. However, the success of liposomes in medical applications has been severely limited by their rapid sequestration in the reticuloendothelial system (RES). Efforts to reduce the RES uptake of liposomes led in the late 1980s to the development of liposomes with a significant increase in their circulation half-lives (sterically stabilized liposomes) (SSL), and revived hopes for their development as drug delivery systems. Two independent laboratories, from studying the biology of red blood cells, identified the presence of sialic acid on the membrane of erythrocytes to be partly responsible for their very long circulation times. Indeed, the incorporation of sialated glycolipids such as the ganglioside GM₁ into phosphatidylcholine (PC):cholesterol (Chol) liposomes effectively increased the circulation time of the vesicles (Allen et al., FEBS Letter 223:42-46 (1987); Allen et al., U.S. Pat. No. 4,920,016, Appl. 132,136, 18 Dec. 1987; 24 pp, 24 Apr. 1990; Gabizon et al., Proc. Natl. Acad. Sci. USA 8:6949 (1988)). These first results have raised new perspectives for liposomes as drug carriers, especially in the field of chemotherapy, since longer half-lives correlated well with higher uptake by implanted tumors in mice (Gabizon et al., Proc. Natl. Acad. Sci. USA 8:6949 (1988)).

In the 1990s, the near simultaneous development by several investigators of the second generation of SSL containing lipid derivatives of polyethylene glycol (PEG) resulted in further improvements (Klibanov et al., FEBS Letter 268 (1):235-237 (1990); Allen et al., Biochim. Biophys. Acta 1066:29-36 (1991)). Klibanov et al., FEBS Letter 268 (1):235-237 (1990) demonstrated that the blood clearance half-life of PC/Chol (1:1) liposomes in mice was 30 min vs. 5 hours for vesicles composed of PC/Chol/PEG-PE (1:1:0.15). Besides, the preparation techniques of the conjugated phospholipid PEG-di-steroyl-phosphatidylethanolamine (DSPE) were reported to be quick and simple (Klibanov et al., FEBS Letter 268 (1):235-237 (1990); Allen et al., Biochim. Biophys. Acta 1066:29-36 (1991), and PEG had already received approval for pharmaceutical use (PEG-ADA, Rhinaris®).

Of interest to the present invention is the observation of increased half-life of circulating protein through conjugation of the protein to a water soluble polymer [Nucci, et al., Adv. Drug Del. Rev. 6:133-151 (1991); Woodle, et al., Proc. Intern. Symp. Control. Rel. Bioact. Mater. 17:77-78 (1990)]. This observation led to the development of sterically stabilized liposomes (SSL) (also known as “PEG-liposomes”) as an improved drug delivery system which has significantly minimized the occurrence of rapid clearance of liposomes from circulation. [Lasic and Martin, Stealth Liposomes, CRC Press, Inc., Boca Raton, Fla. (1995)]. SSL are polymer-coated liposomes, wherein the polymer, preferably polyethylene glycol (PEG), is covalently conjugated to one of the phospholipids and provides a hydrophilic cloud outside the vesicle bilayer. This steric barrier delays the recognition by opsonins, allowing SSL to remain in circulation much longer than conventional liposomes [Lasic and Martin, Stealth Liposomes, CRC Press, Inc., Boca Raton, Fla. (1995); Woodle, et al., Biochem. Biophys. Acta 1105:193-200 (1992); Litzinger, et al., Biochem. Biophys. Acta 1190:99-107 (1994); Bedu Addo, et al., Pharm. Res. 13:718-724 (1996)] and increases the pharmacological efficacy of encapsulated agents, as demonstrated for some chemotherapeutic and anti-infectious drugs [Lasic and Martin, Stealth Liposomes, CRC Press, Inc., Boca Raton, Fla. (1995)]. Studies in this area have demonstrated that different factors affect circulation half-life of SSL, and ideally, the mean vesicle diameter should be under 200 mn, with PEG at a molecular weight of approximately 2,000 Da at a concentration of 5% (9-12) [Lasic and Martin, Stealth Liposomes, CRC Press, Inc., Boca Raton, Fla. (1995); Woodle, et al., Biochem. Biophys. Acta 1105:193-200 (1992); Litzinger, et al., Biochem. Biophys. Acta 1190:99-107 (1994); Bedu Addo, et al., Pharm. Res. 13:718-724 (1996)].

The mechanism by which SSL avoids macrophages and circulate longer in the blood is thought to involve the formation of a “steric barrier” around the liposomes by the attached PEG molecules. Torchilin, et al., Stealth Liposomes, D. Lasic and F. Martin (Eds.), CRC Press, Boca Raton, Fla., pp. 51-62 (1995) claimed that the ability of PEG to prevent liposome opsonization is determined by its behavior in the solvent which entails the formation of a hydrophilic cloud over the vesicle surface even at relatively low polymer concentrations. This negative, hydrophilic coat would act as a protective shield delaying the binding of opsonins that are often attracted to the positive charged lipid surfaces.

The circulation time of sterically stabilized liposomes may be controlled by selection of their size, PEG molecular weight, chain length and concentration and selection of the lipid composition. Maruyama et al., Chem. Pharm. Bull. 39:1620-1622 (1991) tested SSL with different PEG molecular weights (1,000, 2,000, 5,000, and 12,000 Da), with a constant size (180 to 200 nm) and composition (6% DSPE-PEG in DSPC/Chol-(1:1)). The PEG_{2,000}-liposomes appeared to be the longest lasting formulation in mice, with 47.1% of injected dose after 6 h still in the blood. Klibanov et al., FEBS Letter 268 (1):235-237 (1990) conducted similar studies on mice with PC/Chol/PEG-PE (10:5:1) extruded liposomes of 200 nm diameters, using PEG₇₅₀, PEG_{2,000} and PEG_{5,000}. The authors evaluated the “degree of steric barrier” produced on the liposome surface and concluded that it was directly correlated to chain length of PEG and concentration-dependent. They suggested that the SSL prolongation was directly proportional to PEG chain length, which, itself, corresponded to the steric barrier. Finally, other groups (Allen et al., Biochim. Biophys. Acta 1066:29-36 (1991); Woodle et al., Biochim. Biophys. Acta 1105:193-200 (1992)) found somewhat contradictory results showing that the extension of PEG chain length from 2,000 to 5,000 Da had no additional suppression effect on RES uptake. PEG of molecular weights 1,900, 2,000 and 5,000 have been recently used in various applications.

Huang's group (Kibanov et al., Biochim. Biophys. Acta 1062:142-148 (1991); Litzinger et al., Biochim. Biophys. Acta 1190:99-107 (1994) pointed out the importance of the size of liposomes in biodistribution studies, and observed that small vesicles (<100 nm) were taken up by the liver, whereas larger ones (300 nm>diameter<500 nm) accumulated in the spleen, particularly in the red pulp and marginal zone. Indeed, the major function of the spleen is to filter the aged or damaged red blood cells, and the liposome uptake was shown to use this same filtration mechanism, followed by splenic macrophage endocytosis. The reason for such an uptake is however unknown. Their studies showed an optimized circulation time for SSL of 150-200 nm diameters. Ghosh et al., Stealth Liposomes, D. Lasic and F. Martin (Eds.), CRC Press, Boca Raton, Fla. pp. 13-24 (1995) confirmed this work, showing the limitation of the prolongation effect of SSL to a narrow size range, between 70 and 200 nm diameter. Most of SSL applications seem indeed to include a size reduction step in their liposome preparation methods.

Klibanov et al., Biochim. Biophys. Acta 1062:142-148 (1991) studied the effect of the lipid composition on the blood circulation time of SSL, and found that the half-lives of different SSL were all very close, except when phosphatidylserine (PS) was added. Woodle et al., Biochim. Biophys. Acta 1105:193-200 (1992) also conducted biodistribution studies on mice and rats with SSL of various lipid compositions. They showed similarly that an increase in the hydrogenation of PC (i.e. bulk lipid transition temperature), the addition of the anionic lipid PG, and different levels of cholesterol had no impact on the prolongation effect. A consistent half-life of about 15 hours for blood clearance was observed, regardless of the phospholipid's phase transition, cholesterol content or neutral/negative charges.

Nevertheless, Bedu-Addo et al., Pharm. Res. 13:718-724 (1996) recently shed light on the role of cholesterol in the stabilization of liposomes, claiming that the most suitable formulation for prolonged circulation times should contain a minimum of 30 mol % cholesterol, with low concentrations of short-chain PEG-PE (<10%). The authors investigated the efficiency of surface protection in vitro using a fluorescence energy transfer technique. The addition of cholesterol improved surface protection, due to the increase in bilayer cohesive strength. It would limit the formation of “bald spots” less enriched with PEG-PE in the liposomal bilayer, thus inhibiting phase separation and lipid exchange with blood lipoproteins. However, in vivo, it was shown that the long-lasting circulation of SSL seems to depend mostly on the PEG coating and less on the liposome bilayer composition.

Different investigators reported that only 5% PEG-PE could give an optimized steric barrier effect on the vesicles (Klibanov et al., Biochim. Biophys. Acta 1062:142-148 (1991); Woodle et al., Biochim. Biophys. Acta 1105:193-200 (1992); McIntosh et al., Stealth Liposomes, D. Lasic and F. Martin (Eds.), CRC Press, Boca Raton, Fla. pp. 63-71 (1995)). A maximal limit of 10 mol % PEG was very recently proposed to obtain adequate results from in vitro studies, because of the spontaneous formation of micelles of PEG-PE at higher concentrations (Bedu-Addo et al., Pharm. Res. 13:718-724 (1996)).

Also of interest to the present application is the disclosure of PCT Application PCT/US97/05161 relating to improvements in sterically stabilized liposomes and therapeutic and diagnostic including acoustic diagnostic methods of using same.

Of interest to the present invention is work relating to molecular aggregates called “micelles” which are defined as colloidal aggregates spontaneously formed by amphiphilic compounds in water above a critical solute concentration, the critical micellar concentration (CMC), and at solution temperatures above the critical micellar temperature (CMT). The molecules constituting the micelles are in rapid dynamic equilibrium with the unassociated molecules. The increase in the concentration above the CMC usually leads to an increase in the number of micelles without any change in micellar size; however, in certain cases with phospholipid mixed micelles, the spherical micelles enlarge into rod-shaped micelles (Carey et al., Arch. Inter Med. 130:506-527 (1972); Hjelm, Jr. et al., J. Phys. Chem. 96 (21):8653-8661 (1992)). The CMC is strongly temperature dependent, and at a given concentration the monomer to micelle transition occurs gradually over a broad temperature range (Almgren et al., Colloid Polym. Sci. 273:2-15 (1995)). An increase in the temperature leads to an increase in the number of aggregates, while the hydrodynamic radius remains constant (Nivaggioli et al., Langmuir. 11 (3):730-737 (1995); Alexandridis et al., Langmuir. 11:1468-1476 (1995)). In general the increase in temperature leads to an increase in hydrophobic interactions and the water dielectric constant is reduced augmenting the ionic repulsion forces. There are many ways to determine the CMC of an amphiphilic compound (surface tension measurements, solubilization of water insoluble dye, or a fluorescent probe, conductivity measurements, light scattering, and the like). According to a preferred method, surface tension measurements may be used to determine the CMC of PEG-DSPE micelles at room temperature.

Surfactant micelles are used as adjuvants and drug carrier systems in many areas of pharmaceutical technology. Micelles have been used to increase bioavailability or decrease adverse effects of the drugs (Trubetskoy et al., Advan. Drug Deliv. Reviews 16:311-320 (1995). In addition, the small size of micelles play a key role in transport across membranes including the blood brain barrier (Muranushi et al., Chemistry and Physics of Lipids 28:269-279 (1981); Saletu et al., Int. Clin. Psychopharmacol. 3:287-323 (1988). The surfactant micelles are thermodynamically unstable in aqueous media and subject to dissociation upon dilution. Yokoyama et al., Makromol Chem. Rapid Commun. 8:431-435 (1987) proposed a class of amphiphilic polymers, such as polyethylene glycol (PEG), which are known to form more stable polymeric micelles in aqueous solutions. There are many advantages to polymeric micelles, such as small size might control penetration across physiological barriers, increases the half-life in vivo, and allows to target micelles to specific tissues.

Studies involving polymer conjugated lipid micelles, such as PEG conjugated to PE are very recent. In one such study, where polyethylene-oxide (PEO) is conjugated to PE and dissolved in aqueous media forming micelles. The study performed by Trubetskoy et al., Acad. Radiol. 3:232-238 (1996) used PEO-PE conjugated lipid to encapsulate indium-111 and gadolinium chalets as contrast media for precutaneous lymphography using magnetic resonance imaging (MRI) topography. The study concluded that PEO-PE micelles can incorporate amphiphilic agents and prolong their actions in vivo by avoiding the RES, and prolonging the circulation period.

The stability of amphiphilic micelles depends on the strength of Van der Waals interactions. The polymer presence on the micellar surface contributes to its steric protection by repulsive action of the hydrophilic layer from the hydrophobicity of macrophages, thus decreasing the uptake by reticuloendothelial system (RES). Furthermore, the negative charge of the polymer creates a repulsive steric effect in vivo that prevents the binding of opsonins, plasma protein that facilitates RES uptake (Trubetskoy et al., Proceed. Intern. Symp. Control. Rel. Bioact. Mater. 22:452-453 (1995)). Thus, the polar and electrostatic interactions of the polymer with the in vivo environment is responsible for the steric stabilization of phospholipid micelles in vivo.

For sterically stabilized phospholipid micelles (SSM) formation an optimal amphiphilic compound is required, one with the right amount of hydrophobicity and hydrophilicity. Factors such as molecular weight and chain length of polymer, size, lipid concentration, and polymer concentrations may play a very important role in determination of the optimal micellar formulation. However, so far there have been no phospholipid micelles studies performed that evaluate the parameters for optimal formulation and activity.

Conversely, many studies of block copolymer, amphiphilic polymers, micelles have been done. Nivaggioli et al., Langmuir. 11 (3):730-737 (1995) tested block copolymer micelles of different pluronic copolymers (PEO-PPO-PEO) at a constant temperature and concentrations. The authors found that the increase in the molecular weight of the copolymer leads to an increase in the hydrodynamic size, thus suggesting an increase in the hydrophobic core size. Thus, the increase in micelle size due to the molecular weight and chain length would lead to an increase in uptake by RES. Therefore, high molecular weight and chain length decreases circulation time and hence the half-life of the SSM. Overall, the authors found PEO to be the most promising copolymer for SSM stability. Moreover, Carey and co-workers have determine that significant increase in the polymer concentration above the CMC leads to the formation of rod-like micelles causing an increase in the viscosity of the solution (Carey et al., Arch. Inter Med. 130:506-527 (1972); Almgren et al., Colloid Polym. Sci. 273:2-15 (1995)). Therefore, the elongated micelles increase the hydrophobicity of the micelles and may allow more of the non-polar drug to be encapsulated.

From these block co-polymers, amphiphilic compounds, one can infer that the study of parameters that optimize the formulation and activity of phospholipid micelle stability to be very relevant, and should be considered in the future.

The utilization of SSM as drug delivery system is a fairly new application, especially as therapeutic and diagnostic agents. As Trubetskoy et al., Proceed. Intern. Symp. Control. Tel. Bioact. Mater. 22:452-453 (1995) pointed out, almost every possible drug administration route has benefitted from the use of micellar drug formation in terms of increased bioavailability or reduced adverse effects. The small size of the micellar formulation allows for their penetration of blood brain barrier making it an ideal carrier for treatment of CNS diseases, such as Alzheimer's disease. Recently, SSM have been used as diagnostic agents using MRI and STM techniques (Trubetskoy et al., Proceed. Intern. Symp. Control. Test. Bioact. Mater., 22:452-453 (1995); Zareie et al., Collids and Suraces A: Physiocochemical and Engineering Aspects. 112:19-24 (1996)). In both cases SSM were incorporation with either a dye or paramagnetic agents followed by parenteral administration and visualization. In both cases the half-life of the SSM was at least 2 hours.

Also of interest to the present invention is the disclosure of Friedman et al., U.S. Pat. No. 5,514,670 which relates to submicron emulsions for delivery of bioactive peptides including vasoactive intestinal peptide analog. The submicron particles are said to have a weighted average diameter of 10 to 600 nm, more preferably 30 to 500 nm and most preferably 70 to 300 nm.

Of further interest to the present invention is calmodulin (CaM) which is an ubiquitous 17 kd protein that is found widely in the body and has many functions. Calmodulin functions mainly as a regulatory protein and serves as a sensor for calcium ions. The binding of calcium ions (Ca⁺²) to four sites in calmodulin induces the formation of α-helix and other conformational changes that convert it from an inactive to an active form. The activated calmodulin in turn binds to many enzymes and proteins in the cell and modifies their activity. The globular structure of CaM hides hydrophobic binding sites for proteins that are exposed upon CaM interactions with Ca⁺² ions and/or membrane phospholipids (Chiba et al., Life Sciences 47:953-960 (1990); Damrongehai et al., Bioconjugate Chem., 6:264-268 (1995)). Bolin, Neurochem. Int. 23:197-214 (1993) found that VIP is a potent stimulant of Ca⁺² binding to calmodulin suggesting a correlation of VIP interactions with CaM and specific cellular regulatory activities.

Paul et al., Neurochem. Int. 23:197-214 (1993) also reported that internalized VIP had the ability to directly bind to calmodulin (CaM), and that it inhibited both phosphodiesterase as well as the calmodulin-dependent myosin light chain kinase activity. This observation supports a functional role for VIP-CAM complex (Stallwood et al., J. Biol. Chem. 267:19617-19621 (1992); Shiraga et al., Biochem. J. 300:901-905 (1994), therefore suggesting that calmodulin, a multifunctional protein responsible for the regulation of many different signaling enzymes, could be an intracellular receptor for VIP (Paul et al., Neurochem. Int. 23:197-214 (1993). Thus, VIP may regulate signal transduction by CaM association. Moreover, CaM is also found in extracellular fluid and cerebrospinal fluid and that it is actively secreted by cells (Paul et al., Neurochem. Int. 23:197-214 (1993)), thus the VIP-CaM complex may protect the peptide from protease digestion. Ca⁺² ions and lipids are known to effect the peptide-CaM interactions. VIP and Ca⁺² binding by CaM is cooperative, in that calcium ion binding to receptors facilitates VIP binding to CaM and vise versa. Phospholipase treatment has been shown to inhibit VIP binding in intact membranes and modulates the binding by solubilizing VIP-binding protein fractions (Paul et al., Ann. N. Y. Acad. Sci. 527:282-295 (1988)). Thus, the biochemical consequences of VIP-CaM binding depends on the identity of CaM binding site, and conformational changes induced by VIP-CaM binding.

Thus, there exists a need in the art to provide further improvements in the use of micelle technology for the therapeutic and diagnostic administration of bioactive molecules particularly in the treatment of specific disease states. More specifically, there remains a desire in the art for improved methods for administration of amphipathic peptides including, but not limited to, members of the VIP/GRF family of peptides associated with phospholipids in order to achieve a more prolonged and effective therapeutic effect.

### SUMMARY OF THE INVENTION

The present invention provides improved methods of preparing biologically active micelle products comprising one or more biologically active amphipathic compounds in association with a micelle. As used herein, compounds embrace peptides, proteins, enzymes in general, as well as fragments, analogs, and modulators thereof. With respect to polypeptides, the invention contemplates use of both L and D forms. Where compounds of the invention exist in both cis and trans conformations, the invention comprehends use of either form alone or a combination of both forms. The micellar formulations of the invention deliver and enhance bioactivity of the biologically active peptides in a manner which provides improvements in the efficacy and duration of the biological effects of the associated peptides. Increased efficacy and duration of the biological effect is believed to result, at least in part, from interaction of the compound with the micelle in such a manner that the compound attains, and is maintained in, an active or more active conformation than the compound in an aqueous environment. The invention thus overcomes the problems associated with previous liposomal formulations, such as, but not limited to, uptake by the reticuloendothelial system, degradation of the compound, or delivery of the compound in an inactive conformation. According to one aspect of the present invention, polyethylene-glycol (PEG) is covalently conjugated to DSPE and used to form polymeric micelles which are then passively loaded with VIP. The PEG-DSPE forms micelles with a hydrophobic core consisting of distearoyl phosphatidylethanolamine (DSPE) fatty acid chains which is surrounded by a hydrophilic “shell” formed by the PEG polymer.

According to one aspect of the invention, a method is provided for preparing a biologically active micelle product comprising one or more biologically active amphipathic compounds in association with a micelle; said method comprising the steps of a) mixing a combination of one or more lipids wherein said combination includes at least one lipid component covalently bonded to a water-soluble polymer; b) forming sterically stabilized micelles from said combination of lipids; and c) incubating micelles from step b) with one or more biologically active amphipathic compounds under conditions in which said compound become associated with said micelles from step b) in a more biologically active conformation as compared to the compound in an aqueous solution. According to a further aspect of the invention, a biologically active micelle product may be produced by the coprecipitation of a biologically active amphipathic compound with lipids to form micelles with incubation not required. Specifically, a method is provided of preparing a biologically active micelle product comprising one or more biologically active amphipathic compound in association with a micelle; said method comprising the steps of: a) mixing one or more lipids wherein said combination includes at least one lipid component covalently bonded to a water-soluble polymer with a biologically active amphipathic compound; b) forming sterically stabilized micelles from the mixture of step (a) under conditions in which said compound becomes associated with said micelles in an active conformation.

As one aspect of the invention, the micelles are sterically stabilized micelles (SSM) which are produced from a combination of lipids which includes at least one lipid component covalently bonded to a water-soluble polymer. This polymer bound phospholipid is the micelle forming component. Other lipids are actually solubilized in this micelle to form mixed micelles. The water-soluble polymer, which is preferably polyethylene glycol (PEG) increases the lipid solubility to form micelles instead of vesicles in aqueous media. It also acts to sterically stabilize the resulting micelle against uptake by components of the reticuloendothelial system.

In another aspect, the invention provides a method for preparing a biologically active sterically stabilized micelle product comprising one or more biologically active amphipathic compounds, said method comprising the steps of: a) preparing a mixture of an aqueous solution with one or more lipids wherein at least one lipid is conjugated to a water soluble polymer; b) forming sterically stabilized micelles; c) mixing said micelles with one or more amphipathic compound(s); d) incubating said micelles and said amphipathic compound(s) under conditions wherein the amphipathic compound(s) assumes a more favorable biologically active conformation upon association with said micelle as compared to the compound in an aqueous solution.

In another aspect, the invention provides a method for preparing a biologically active sterically stabilized micelle product comprising one or more biologically active amphipathic compounds, said method comprising the steps of: a) dissolving in an organic solvent one or more lipids wherein at least one lipid is conjugated to a water soluble polymer; b) removing the organic solvent to leave a dry lipid film; c) hydrating the dry lipid film with an aqueous solution; d) forming sterically stabilized micelles; e) combining said micelles with one or more amphipathic compounds; and f) incubating said micelles and said amphipathic compound(s) under conditions wherein the amphipathic compound(s) assumes a more favorable biologically active conformation upon association with said micelle as compared to the compound in an aqueous solution.

In another aspect, the invention provides a method for preparing a biologically active sterically stabilized micelle product comprising one or more biologically active compounds and one or more targeting compounds; said method comprising the steps of: a) dissolving in an organic solvent said biologically active compound(s) and one or more lipids wherein at least one lipid is conjugated to a water soluble polymer; b) removing the organic solvent to leave a dry film; c) hydrating the dry film with an aqueous solution; d) forming sterically stabilized micelle products; e) combining said micelle products with one or more targeting compounds; and f) incubating said micelle products under conditions wherein the targeting compound(s) associates with said micelle products. In one aspect, the targeting compound is linked to one or more lipid components of the micelle. Preferably linkage between the targeting compound and the lipid is effected by covalent means in a manner that permits the targeting compound to interact with its cognate receptor, ligand, or binding partner and position the micelle in close proximity.

The methods of the invention are useful with any biologically active amphipathic compound, peptide, protein, or fragment, analog, or modulator thereof which can thereby be stably maintained in an active conformation in association with or within the lipid core of the micelle. Preferred amphipathic compounds include those characterized by having one or more α- or π-helical domains in their biologically active conformation and particularly those in which polar and apolar residues are separated on opposite sides of the helix. Particularly preferred amphipathic compounds useful with the invention include any member of the vasoactive intestinal peptide (VIP)/growth hormone releasing factor (GRF) family of peptides which includes biologically active analogs thereof. The mammalian and non-mammalian VIP/GRF family of peptides includes functional analogs of VIP and GRF, peptide histidine isoleucine (PHI), peptide histidine methionine (PHM), growth hormone releasing factor (GRF), hypocretins, pituitary adenylate cyclase activating peptide (PACAP), secretin, and glucagon. Like VIP, other members of the VIP/GRF family of peptides, and biologically active analogs thereof, can form amphipathic helices wherein hydrophobic and hydrophilic domains of the peptide are segregated and the hydrophobic domain(s) is capable of binding lipid core. The invention also contemplates the use of other neuropeptides including neuropeptide Y (NPY), neuropeptide YY (NPYY), ACTH, calcitonin, GAP (GnRH precursor molecule), glutamate-decarboxylase, GnRH/GL, keyhole limpet hemocyanin, leucin-enkephalin, mesotocin, methionin-enkephalin, neurotensin, peroxydase, somatostatin, substance P, vasopressin, and vasotocin. The invention also contemplates modulators having enhanced bioactivity in association with micelles prepared by a method of the invention. A particularly preferred peptide for use according to the invention is VIP. In one aspect, micelles according to the invention are characterized by an average diameter of less than about 20 nm. According to one aspect of the invention the micelles further comprise calmodulin. The biologically active peptide products of the invention may be utilized in a wide variety of therapeutic, diagnostic, cosmetic and organ, tissue and cell preservative uses wherein it is desired to deliver a high level of biologically active compound or to detect targeted delivery of the micelle product as will be described below.

In another aspect, the invention provides methods for preparing a biologically active sterically stabilized crystalline product comprising one or more biologically active compounds which are insoluble in an aqueous solution; said method comprising the steps of: a) dissolving in an organic solvent said biologically active compound(s) and one or more lipids wherein at least one lipid is conjugated to a water soluble polymer; b) removing the organic solvent to leave a dry film; c) hydrating the dry film with an aqueous solution; and d) forming a sterically stabilized crystalline product. As used herein and in subsequently described crystalline products of the invention, “insoluble” is defined according to the U.S. Pharmacopeia National Formulary [USP 23, 1995, page 10], as requiring 10,000 or more parts of solvent for 1 part of solute. The crystalline product of the method is essentially a micelle-encased aggregate of the insoluble compound which is densely packed and crystalized.

In still another aspect, the invention provides methods for preparing a biologically active sterically stabilized crystalline product comprising one or more biologically active compounds which are insoluble in an aqueous solution; said method comprising the steps of: a) dissolving in an organic solvent said biologically active compound and one or more lipids wherein at least one lipid is conjugated to a water soluble polymer; b) freeze-drying to remove the organic solvent; c) hydrating with an aqueous solution; and d) forming a sterically stabilized crystalline product.

In still another aspect, the invention provides methods for preparing a biologically active sterically stabilized crystalline product comprising one or more biologically active compounds which are insoluble in aqueous solution and one or more amphipathic targeting compounds; said method comprising the steps of: a) dissolving in an organic solvent said biologically active compound(s) and one or more lipids wherein at least one lipid is conjugated to a water soluble polymer; b) removing the organic solvent to leave a dry film; c) hydrating the dry film with an aqueous solution; d) forming sterically stabilized crystalline products; e) combining said crystalline products with one or more targeting compounds; and f) incubating said crystalline products under conditions wherein the targeting compound(s) associates with said crystalline products, said targeting compound conjugated to a lipid of the micelle.

Methods of the invention for producing sterically stabilized crystalline products are amenable to the use of any compound that is insoluble in an aqueous solution. Preferred insoluble compounds include, but are not limited to, progesterone, testosterone, estrogen, prednisolone, prednisone, 2,3 mercaptopropanol, amphotericin B, betulinic acid, camptothecin, diazepam, nystatin, propofol, cyclosporin A, doxorubicin, and paclitaxel (Taxol®), and tetramethyl NDGA. In methods of the invention for producing sterically stabilized crystalline product further comprising one or more targeting compounds, any targeting compound that assumes or maintains a biologically active conformation when in association with the sterically stabilized crystalline product can be used. In a preferred embodiment, any of the amphipathic compounds as described above are utilized. In a most preferred embodiment, the targeting compound is VIP or other member of the VIP/GRF family or proteins. In other embodiments, the targeting compound can be heliospectins I or II or any member of the neuropeptide family such as neuropeptide Y (NPY), neuropeptide YY (NPYY), including neuropeptide fragments 2-36 and related fragments, ACTH, calcitonin, GAP (GnRH precursor molecule), glutamate-decarboxylase, keyhole limpet hemocyanin, leucin-enkephalin, mesotocin, methionin-enkephalin, neurotensin, peroxydase, somatostatin, substance P, vasopressin, and vasotocin.

Composition comprising the biologically active micelle product of the invention include those wherein the biologically active amphipathic peptide, protein, fragment, analog, or modulators thereof has an activity selected from the group consisting of anti-oxidant activity, anti-pain, anti-inflammatory, wound healing activity, anti-microbial, antibronchospasm, metabolic activity, anti-cancer activity, cardiovascular activity, antiglaucoma activity, anti-apoptosis, anti-wrinkling activity, cryopreservation, and anti-aging activity. Compositions of the invention include cosmetic, therapeutic and diagnostic compositions. In the case of diagnostic compositions, the micelle product further comprises a detectable label selected from the group consisting of a fluorescent label, a radioactive label, a dye, a gas, and a compound which enhances radiographic, magnetic resonance, and ultrasound imaging.

The invention further provides methods of treating a pathology selected from the group consisting of autism, amyotrophic lateral sclerosis, multiple sclerosis, eneuresis, Parkinson's disease, brain ischemia, stroke, cerebral palsy (CP) sleeping disorders, feeding disorders, and AIDS-associated dementias comprising the step of administering to an individual suffering from the pathology an amount of a micelle composition effective to inhibit conditions associated with the pathology said micelle composition prepared by a method of comprising the steps of: (a) mixing one or more lipids wherein at least one lipid component is covalently bonded to a water-soluble polymer; (b) forming sterically stabilized micelles from lipids; (c) incubating micelles from step (b) with one or more biologically active amphipathic compound(s) under conditions in which said compound(s) becomes associated with said micelles in a more biologically active conformation, wherein at least one amphipathic compound is a member of the VIP/glucagon/secretin family of peptides including peptide fragments and analogs. In another embodiment, methods of treatment include those wherein in the method of preparing the micelle composition, mixing in step (a) is carried out in an organic solvent, and forming sterically stabilized micelles in step (b) is carried out in steps comprising (i) removing the organic solvent to leave a dry film, and (ii) hydrating the dry film with an aqueous solution. The invention also provides methods of treatment wherein in the method of preparing the micelle composition, the organic solvent in step (a) is removed by evaporation or lyophilization. In one aspect, methods of treating autism, multiple sclerosis, eneuresis, Parkinson's disease, amyotrophic lateral sclerosis, and AIDS-associated dementias according to the invention include those wherein in the method of preparing the micelle composition, mixing in step (a) is carried out in an aqueous solution.

The invention also provides methods of treating a pathology selected from the group consisting of autism, multiple sclerosis, eneuresis, Parkinson's disease, amyotrophic lateral sclerosis, and AIDS-associated dementias comprising the step of administering an amount of a micelle composition effective to alleviate conditions associated with the pathology, said micelle composition prepared in a method comprising the steps of: a) mixing one or more lipids with one or more biologically active amphipathic compounds, wherein at least one lipid component is covalently bonded to a water-soluble polymer, and wherein at least one amphipathic compound is a member of the VIP/glucagon/secretin family of peptides including peptide fragments and analogs; b) forming sterically stabilized micelles from the mixture of step (a) under conditions in which said compound(s) becomes associated with said micelles in a more biologically active conformation. In one aspect, the methods of treatment include those wherein the method of preparing the micelle composition, mixing in step (a) is carried out in an organic solvent and at least one lipid is conjugated to one or more targeting compound(s), and forming micelles in step (b) is carried out in a process comprising the steps of: (i) removing the organic solvent to leave a dry film, and (ii) hydrating the dry film with an aqueous solution, said method further comprising step of: (c) incubating said micelle products under conditions wherein the targeting compound(s) associates with said micelle products in an active conformation.

The invention also provides methods of treating a pathology selected from the group consisting of autism, multiple sclerosis, eneuresis, Parkinson's disease, amyotrophic lateral sclerosis, and AIDS-associated dementias comprising the step of administering to an individual suffering from the pathology an amount of a sterically stabilized crystalline composition effective to inhibit conditions associated with the pathology, said sterically stabilized crystalline composition comprising one or more biologically active compounds which are insoluble in aqueous solution, said sterically stabilized crystalline compounds prepared by a method comprising the steps of: a) mixing the biologically active compound(s) with one or more lipids, wherein at least one of the lipids is conjugated to a water soluble polymer and at least one biologically active compound is a member of the VIP/glucagon/secretin or IL-2 family of peptides including peptide fragments and analogs; and b) forming sterically stabilized crystalline products. In one embodiment, method of the invention include those wherein in the method of preparing the sterically stabilized crystalline compound, mixing in step (a) is carried out in an organic solvent, and forming crystalline products in step (b) is carried out in a process comprising the steps of (i) removing the organic solvent to leave a dry film; and (ii) hydrating the dry film with an aqueous solution, said method further comprising the steps of (c) contacting said crystalline products with one or more targeting compounds; and (d) incubating said crystalline products under conditions wherein the targeting compound(s) associates with said crystalline products. In another aspect, methods of treating autism, multiple sclerosis, eneuresis, Parkinson's disease, amyotrophic lateral sclerosis, and AIDS-associated dementias according to the invention include those wherein in the method of preparing the sterically stabilized crystalline compound, forming in step(b) is carried out in the steps comprising (i) removing the organic solvent to leave a dry film and (ii) hydrating the dry film with an aqueous solution.

In one embodiment, method of treating a pathology selected from the group consisting of autism, multiple sclerosis, eneuresis, Parkinson's disease, amyotrophic lateral sclerosis, and AIDS-associated dementias according to the invention include micelle compositions or crystalline compounds wherein the water soluble polymer is polyethylene glycol (PEG). In another embodiment, methods of the invention include use of micelles having an average diameter of less than about 25 nm. In another aspect, methods of the invention include use of micelles having an average diameter of less than about 50 nm. In still another embodiment, methods of the invention include micelle compositions or crystalline compounds wherein the combination of lipids consists of distearoyl-phosphatidylethanolamine covalently bonded to PEG (PEG-DSPE).

The invention further provides a medicament for treating autism, multiple sclerosis, eneuresis, Parkinson's disease, amyotrophic lateral sclerosis, e and AIDS-associated dementias comprising a micelle composition prepared by a method of comprising the steps of: (a) mixing one or more lipids wherein at least one lipid component is covalently bonded to a water-soluble polymer; (b) forming sterically stabilized micelles from lipids; (c) incubating micelles from step (b) with one or more biologically active amphipathic compound(s) under conditions in which said compound(s) becomes associated with said micelles in a more biologically active conformation, wherein at least one amphipathic compound is a member of the VIP/glucagon/secretin or IL-2 family of peptides including peptide fragments and analogs. In another embodiment, medicaments of the invention include those wherein in the method of preparing the micelle composition, mixing in step (a) is carried out in an organic solvent, and forming sterically stabilized micelles in step (b) is carried out in steps comprising (i) removing the organic solvent to leave a dry film, and (ii) hydrating the dry film with an aqueous solution. The invention also provides medicaments wherein in the method of preparing the micelle composition, the organic solvent in step (a) is removed by evaporation or lyophilization. In one aspect, medicaments of the invention include those wherein in the method of preparing the micelle composition, mixing in step (a) is carried out in an aqueous solution.

The invention also provides medicaments for the treatment autism, multiple sclerosis, eneuresis, Parkinson's disease, amyotrophic lateral sclerosis, and AIDS-associated dementias comprising a micelle composition prepared in a method comprising the steps of: a) mixing one or more lipids with one or more biologically active amphipathic compounds, wherein at least one lipid component is covalently bonded to a water-soluble polymer, and wherein at least one amphipathic compound is a member of the VIP/glucagon/secretin family of peptides including peptide fragments and analogs; b) forming sterically stabilized micelles from the mixture of step (a) under conditions in which said compound(s) becomes associated with said micelles in a more biologically active conformation. In one aspect, the medicaments include those wherein the method of preparing the micelle composition, mixing in step (a) is carried out in an organic solvent and at least one lipid is conjugated to one or more targeting compound(s), and forming micelles in step (b) is carried out in a process comprising the steps of: (i) removing the organic solvent to leave a dry film, and (ii) hydrating the dry film with an aqueous solution, said method further comprising step of: (c) incubating said micelle products under conditions wherein the targeting compound(s) associates with said micelle products in an active conformation.

The invention also provides medicaments for the treatment of autism, multiple sclerosis, eneuresis, Parkinson's disease, amyotrophic lateral sclerosis, and AIDS-associated dementias comprising a sterically stabilized crystalline composition comprising one or more biologically active compounds which are insoluble in aqueous solution, said sterically stabilized crystalline compounds prepared by a method comprising the steps of: a) mixing the biologically active compound(s) with one or more lipids, wherein at least one of the lipids is conjugated to a water soluble polymer and at least one biologically active compound is a member of the VIP/glucagon/secretin of IL-2 family of peptides including peptide fragments and analogs; and b) forming sterically stabilized crystalline products. In one embodiment, medicaments of the invention include those wherein in the method of preparing the sterically stabilized crystalline compound, mixing in step (a) is carried out in an organic solvent, and forming crystalline products in step (b) is carried out in a process comprising the steps of (i) removing the organic solvent to leave a dry film; and (ii) hydrating the dry film with an aqueous solution, said method further comprising the steps of (c) contacting said crystalline products with one or more targeting compounds; and (d) incubating said crystalline products under conditions wherein the targeting compound(s) associates with said crystalline products. In another aspect, medicaments according to the invention include those wherein in the method of preparing the sterically stabilized crystalline compound, forming in step(b) is carried out in the steps comprising (i) removing the organic solvent to leave a dry film and (ii) hydrating the dry film with an aqueous solution.

In one embodiment, medicaments of the invention include micelle compositions or crystalline compounds wherein the water soluble polymer is polyethylene glycol (PEG). In another embodiment, medicaments of the invention include use of micelles having an average diameter of less than about 25 nm. In still another embodiment, medicaments of the invention include micelle compositions or crystalline compounds wherein the combination of lipids consists of distearoyl-phosphatidylethanolamine covalently bonded to PEG (PEG-DSPE).

The invention also provides a method of treating a pathology selected from the group consisting of immune disorders, inflammatory conditions, and cancer comprising the step of administering to an individual suffering from the pathology an amount of a micelle composition effective to ameliorate conditions associated with the pathology, said micelle composition prepared by a method of comprising the steps of:

In still another aspect, the invention provides a method of treating a pathology selected from the group consisting of Hashimoto's thyroiditis, pernicious anemia, Addison's disease, diabetes, systemic lupus erythematosus, dermatomyositis, Sjogren's syndrome, dermatomyositis, multiple sclerosis, myasthenia gravis, Reiter's syndrome, Graves disease, inflammatory bowel disease, osteoarthritis, rheumatoid arthritis, asthma, allergies, inflammatory neuropathies (Guillain Barré, inflammatory polyneuropathies), vasculitis (Wegener's granulomatosus, polyarteritis nodosa), and rare disorders such as polymyalgia rheumatica, temporal arteritis, Sjogren's syndrome, Bechet's disease, Churg-Strauss syndrome, and Takayasu's arteritis.

In yet another aspect, the invention provides a method of preventing VIP-induced hypotension comprising the step of administering to an individual an amount of a sterically stabilized micelle or crystalline composition effective to treat a target pathology, said sterically stabilized micelle or crystalline composition prepared by any one of the methods herein.

### DESCRIPTION OF THE DRAWINGS

FIG. 1 depicts surface tension measurements of a PEG-DSPE aqueous solution to determine the critical micelle concentration (CMC) at room temperature;

FIG. 2 depicts the CD spectral analysis of VIP in saline, Hepes buffer, and phospholipids at room temperature;

FIG. 3 depicts the CD spectral analysis of VIP at room temperature and at 37°;

FIG. 4 depicts the effect of calmodulin on the CD spectral analysis of VIP in saline and phospholipids;

FIG. 5 depicts the CD spectral analysis of VIP fragments in saline and phospholipids;

FIG. 6 depicts the CD spectral analysis of VIP and vasopressin (VP) in saline and phospholipids;

FIG. 7 depicts the effect of VIP-SSM on vasodilation; and

FIG. 8 depicts the effect of calmodulin on VIP-SSM induced vasodilation.

### DETAILED DESCRIPTION OF THE INVENTION

The present invention provides improved methods of preparing biologically active micelle products comprising biologically active amphipathic compounds in association with a micelle. The invention also provides method for preparing sterically stabilized crystalline products comprising compounds that are insoluble in an aqueous solution. The crystalline products of the invention are prepared alone or in combination with a targeting compound. It is preferred that the targeting compound is an amphipathic compound that assumes a more favorable biological conformation in association with the crystalline product. The preferred amphipathic compounds are characterized by having hydrophilic and hydrophobic domains segregated to the extent that the hydrophobic domain is capable of associating within the micellar core. Compounds of the invention preferably attain a biologically active conformation in association with or within the micelle core. More biologically active conformations are those in which the desired compound is most likely to be capable of effecting its normal biological activity, for example, through receptor or ligand recognition and binding, and comparison of biological activity is made with respect to the compound in association with the micelle or crystalline product of the invention compared to the compound in an aqueous solution or environment. Compounds of the invention may be characterized by having one or more discrete α- or π-helical domains which segregate the hydrophobic and hydrophilic domains. Preferred compounds of the invention are members of the VIP/GRF peptide family. The most preferred compound of the invention is a member of the VIP/glucagon/secretin or IL-2 family of peptides including peptide fragments and analogs. While biologically active compounds are associated with the micelle core, the association is not irreversible and the compound may be released either quickly or over time from association with the micelle, depending on properties of the micelle and the compound.

In methods of the invention to prepare sterically stabilized crystalline products, any compound that is insoluble in as aqueous solution can be incorporated into crystalline product. In methods of the invention, the insoluble compounds associate in the hydrophobic core of the associated lipids to the extent that the insoluble compound crystallizes. While the invention contemplates the use of any insoluble compound to produce the crystalline products, preferred compounds are normally insoluble anti-cancer agents, antifungal agents, sedatives, and steroidal compounds. Most preferably, the insoluble compounds are selected from the group consisting of paclitaxel (Taxol®), betulinic acid, doxorubicin, amphotericin B, diazepam, nystatin, propofol, testosterone, estrogen, prednisolone, prednisone, 2,3 mercaptopropanol, and progesterone.

Of particular interest to the present invention are the biologically active amphipathic peptides which are members of the family of peptide compounds including, but not limited to, vasoactive intestinal peptide (VIP), growth hormone releasing factor (GRF), hypocretins, peptide histidine isoleucine (PHI), peptide histidine methionine (PHM), pituitary adenylate cyclase activating peptide (PACAP), gastric inhibitory hormone (GIP), hemodermin, the growth hormone releasing hormone (GHRH), sauvagine and urotensin I, secretin, glucagon, galanin, endothelin, calcitonin, α₁, -proteinase inhibitor, angiotensin II, corticotropin releasing factor, antibacterial peptides and proteins in general, surfactant peptides and proteins, α-MSH, adrenolmedullin, ANF, IGF-1, α2 amylin, orphanin, and orexin.

Other peptides of interest include neuropeptides, which serve as integrative chemical messengers, conveying information from one discrete neuronal population to another. Furthermore, it is becoming evident that neuropeptides are involved in coupling transductive events from neurons to glial and to immune cells. Major areas of neuropeptide research encompass pain and analgesia, appetite control, inflammation, mood and affective behavior. In addition to the neuropeptides discussed herein, other neuropeptides include, but are not limited to, heliospectins I or II, neuropeptide Y (NPY), neuropeptide YY (NPYY), including neuropeptide fragments 2-36 and related fragments, ACTH, calcitonin, GAP (GnRH precursor molecule), glutamate-decarboxylase, keyhole limpet hemocyanin, leucin-enkephalin, mesotocin, methionin-enkephalin, neurotensin, peroxydase, somatostatin, substance P, vasopressin, and vasotocin.

Micelles according to the invention may be produced from combinations of lipid materials well known and routinely utilized in the art to produce micelles and including at least one lipid component covalently bonded to a water-soluble polymer. Lipids may include relatively rigid varieties, such as sphingomyelin, or fluid types, such as phospholipids having unsaturated acyl chains. The lipid materials may be selected by those of skill in the art in order that the circulation time of the micelles be balanced with the drug release rate. To make full use of the power of these micelles in drug delivery, a key challenge is to prevent the leakage of the drug from the micelle to a level significantly less than the plasma distribution rate. However, this point is probably the fundamental basis of SSL and SSM, since their delivery, which is difficult to control, corresponds to the bioavailability of the encapsulated agent. SSM being more dynamic than liposomes may show superiority to SSL with respect to drug release. Polymers of the invention may thus include any compounds known and routinely utilized in the art of sterically stabilized liposome (SSL) technology and technologies which are useful for increasing circulatory half-life for proteins, including for example polyvinyl alcohol, polylactic acid, polyglycolic acid, polyvinylpyrrolidone, polyacrylamide, polyglycerol, polyaxozlines, or synthetic lipids with polymeric headgroups. The most preferred polymer of the invention is PEG at a molecular weight between 1000 and 5000. Preferred lipids for producing micelles according to the invention include distearoyl-phosphatidylethanolamine covalently bonded to PEG (PEG-DSPE) alone or in further combination with phosphatidylcholine (PC), and phosphatidylglycerol (PG) in further combination with cholesterol (Chol) and/or calmodulin.

Methods of the invention for preparation of sterically stabilized micelle products or sterically stabilized crystalline products can be carried using various techniques. In one aspect, micelle components are mixed in an organic solvent and the solvent is removed using either evaporation or lyophilization. Removal of the organic solvent results in a lipid film, or cake, which is subsequently hydrated using an aqueous solution to permit formation of micelles. The resulting micelles are mixed with an amphipathic compound of the invention whereby the amphipathic compound associates with the micelle and assumes a more favorable biologically active conformation.

In a more simplified preparation technique, one or more lipids are mixed in an aqueous solution after which the lipids spontaneously form micelles. The resulting micelles are mixed with an amphipathic compound which associates with the micelle products and assumes a more favorable biologically active conformation. Preparing micelle products by this method is particularly amenable for large scale and safer preparation and requires a considerable shorter time frame than methods previously described. The procedure is inherently safer in that use of organic solvents is eliminated.

In methods of the invention for preparing sterically stabilized crystalline products, it is preferred that one or more lipid compounds are mixed in an organic solvent with one or more insoluble compounds. The organic solvent is removed either by evaporation or lyophilization to provide a film, or cake. The resulting film, or cake, is then hydrated by introduction of an aqueous solution. As a result, the insoluble compound associates within the hydrophobic core of the lipid structure and is solubilized or re-crystallizes. In one aspect of the invention, the solubilized compound or crystalline product is mixed with a targeting compound which, as described above for preparation of micelle products of the invention, associates with the crystalline product in a more favorable biologically active conformation. Crystalline products of the invention provide advantages in that they, like sterically stabilized micelle products, are able to evade the RES. More importantly, the crystalline products of the invention permit administration of higher concentrations of the insoluble compound in a small volume, preferable in a size less than 300 nm. The crystalline products also provide a method wherein insoluble compounds, which are normally difficult to effectively administer because of their inherent insolubility, can be effectively administered to a mammal in need thereof.

The micelles and crystalline products produced according to the methods of the invention are characterized by improved stability and biological activity and are useful in a variety of therapeutic, diagnostic and/or cosmetic applications. According to one embodiment, the invention comprehends a composition comprising a biologically active micelle product wherein said biologically active amphipathic compound has anti-oxidant activity, anti-aging, anti-wrinkle formation or wound healing capacity. Compositions of this type may be of cosmetic or therapeutic nature. The preferred cosmetic composition includes a biologically active member of the VIP/glucagon/secretin family of peptides including peptide fragments and analogs. The invention also provides an oral controlled release preparation for the treatment of a gastrointestinal disorder wherein said preparative method further comprises the step of encapsulating the biologically active micelle or crystalline product in an enteric coated capsule. Alternatively, the micelle or crystalline product may be encapsulated in a gelatin capsule. The oral controlled release preparation is useful in a variety of gastrointestinal disorders including those selected from the group consisting of inflammatory bowel disease, chronic constipation, Hirschprung's disease, achalasia, infantile hypertrophic pyloric stenosis, and ulcers. Other indications for use of the micelles of the invention, particularly those micelles containing a member of the VIP/GRF family of proteins, peptides and fragments, analogs, and modulators, include asthma, chronic obstruction pulmonary disease, arthritis, lupus erythematosus, Alzheimer's disease, cerebralpalsy, stroke, glaucoma, acute food impaction, scleroderma, rhinitis, systemic and pulmonary hypertension, psoriasis, baldness, autism, multiple sclerosis, eneuresis, Parkinson's disease, amyotrophic lateral sclerosis, and AIDS-associated dementias, impotence and female arousal sexual dysfunction. The preferred oral preparation includes a biologically active member of the VIP/glucagon/secretin or IL-2 family of peptides including peptide fragments and analogs. Micelle preparations comprising a biologically active member of the VIP/glucagon/secretin or IL-2 family of peptides including peptide fragments and analogs are also a promising therapeutic agent for conditions such as asthma, chronic obstruction pulmonary disease, systemic and pulmonary hypertension, scleroderma, cystic fibrosis, bronchiectasis, myocardial ischemia, impotence and baldness. Still other indications include decreased sperm/ova motility, decreased mucociliary clearance, Kartagener's syndrome, increased inflammatory cell migration and activation, increased secretion of mucin, decreased chloride ion secretion (often associated with cystic fibrosis), vasoconstriction, vascular obstruction to an organ or tissue (often associated with sickle vaso-occlusive crisis), constipation, impotence and female sexual arousal dysfunction. The invention further provides methods for cosmetic use and preserving a bodily organ, tissue, or cell type for storage and transplantation or fertilization in a recipient comprising the step of incubating said organ, tissue, or cell in a micelle composition comprising a member of the VIP/glucagon/secretin or IL-2 family of peptides including peptide fragments and analogs.

In still another aspect of the invention, micelle products prepared with or without associated amphipathic compounds can be used to improve viability of cells, tissues, and organs that are stored cryogenically. In this aspect, cells are contacted with a micelle product of the invention prior to cryogenic storage either alone, or in the presence of other storage compounds, e.g., dimethylsulfoxide (DMSO), sucrose, glycerol, or ethylene glycol, well known and routinely used in the art.

The invention further provides methods of administering a biologically active amphipathic compound to a target tissue comprising the steps of: preparing a biologically active micelle or crystalline product comprising a biologically active amphipathic compound in association with a micelle or crystalline product according to the methods of the invention and administering a therapeutically effective amount of the micelle or crystalline product to said target tissue. The micelle products of the invention may be administered intravenously, intraarterially, intranasally such as by aerosol administration, nebulization, inhalation, or insufflation, intratracheally, intra-articularly, orally, transdermally, subcutaneously, topically onto mucous membranes, such as, but not limited to, oral mucosa, lower gastrointestinal mucosa and conjunctiva, and directly onto target tissues. Methods of administration for amphipathic compounds are equally amenable to administration of compounds that are insoluble in aqueous solutions.

Biologically active compounds in therapeutic methods can be administered at significantly reduced dosage levels as compared to administration of the compound alone, particularly wherein the compound has a particularly short half life or lowered bioactivity in circulation. For example, VIP in association with SSM can be expected to exhibit enhanced and prolonged bioactivity in comparison to VIP administered alone. Regardless of which bioactive compound is associated with SSM, the micelle product must be tested in order to determine a biologically effective amount required to achieve the same result effected by the compound administered by conventional means. The worker of ordinary skill in the art would realize that the biologically effective amount of a particular compound when delivered by conventional means would serve as a starting point in the determination of an effective amount of the compound in SSM. It would therefore be highly predictive that the same and lesser dosages in SSM would be effective as well and merely routine to determine the minimum dosage required to achieve a desired biological effect. In the case of VIP administration, for example, if conventional administration would require a dosage of 20 mg, VIP in SSM would likely require significantly less in order to achieve the same effect. As with administration of amphipathic compounds in association with sterically stabilized micelle products, sterically stabilized crystalline (SSC) products permit administration of more effective dosages of compounds that are insoluble in aqueous solutions.

Another aspect of the invention is the means for preventing VIP-induced hypotension. One of the deleterious effects of VIP administration has been the resulting hypotension brought on by vasodilation. Hypotension is an abnormal condition in which the blood pressure is lower than 90/60 or is low enough to cause symptoms or interfere with well-being. Blood pressure is normally above 90/60 mmHg (millimeters of mercury). When the blood pressure is too low there is inadequate blood flow to the heart, brain, and other vital organs. VIP administered in a sterically stabilized micelle provides a means for limiting VIP-induced hypotension.

An exemplary regiment in the treatment, for example, of autism, multiple sclerosis, eneuresis, Parkinson's disease, amyotrophic lateral sclerosis, and AIDS-associated dementias, would include administration of from 0.001 mg/kg body weight to about 1000 mg/kg, from about 0.01 mg/kg to about 100 mg/kg, from about 0.1 mg/kg to about 100 mg/kg, about 1.0 mg/kg to about 50 mg/kg, or from about 1 mg/kg to about 20 mg/kg, given in daily doses or in equivalent doses at longer or shorter intervals, e.g., every other day, twice weekly, weekly, monthly, semi-annually, or even twice or three times daily. Alternatively, dosages may be measured in international units (IU) ranging from about 0.001 IU/kg body weight to about 1000 IU/kg, from about 0.01 IU/kg to about 100 IU/kg, from about 0.1 IU/kg to about 100 IU/kg, from about 1 IU/kg to about 100 IU/kg, from about 1 IU/kg to about 50 IU/kg, or from about 1 IU/kg to about 20 IU/kg. Administration may be oral, intravenous, subcutaneous, intranasal, inhalation, transdermal, transmucosal, or by any other route discussed herein.

Association of a biologically active amphipathic or insoluble compound with SSM or SSC product, respectively, of the invention would be expected to increase the magnitude of the biological effects of the compound from about 50 to 100% over the effects observed following administration of the compound alone. Likewise, association with SSM or SSC of the invention would be expected to invoke a longer lasting biological effect.

The invention further provides improved diagnostic compositions comprising biologically active micelle products and methods for their use comprising the steps of: preparing a biologically active micelle product comprising a biologically active amphipathic compound in association with a micelle prepared according to the methods of the invention; administering a diagnostically effective amount of the micelle product to a target tissue or organ; and detecting uptake or interaction of the micelle product at the target tissue or organ. According to one aspect of the invention, the target tissue is a tumor. In one aspect of the method, the micelle product is detectably labeled with a label selected from the group including a radioactive label, a fluorescent label, a non-fluorescent label, a dye, a gas, or a compound which enhances radiographic, magnetic resonance, and ultrasound imaging (MRI) which label is detected at the target tissue.

The invention also provides use of a biologically active micelle product comprising a biologically active amphipathic compound and produced according to methods of the invention for the treatment of inflammation, chronic obstruction pulmonary disease, increased secretion of mucin, acute food impaction, rhinitis, Kartagener's syndrome, cystic fibrosis, bronchiectasis, hypertension, allergy, Alzheimer's disease, cerebral palsy sleep disorder, stroke, atherosclerosis, inflammatory bowel disorder, chronic constipation, Hirschprung's disease, achalasia, infantile hypertrophic pyloric stenosis, ulcers, to enhance or decrease cell proliferation, prevent apoptosis, to promote wound healing in a body organ or tissue, and to prevent cell, organ, tissue rejection, autism, multiple sclerosis, eneuresis, Parkinson's disease, amyotrophic lateral sclerosis, and AIDS-associated dementias, impotence and female arousal sexual dysfunction. As discussed herein, neonatal blood from autistic children has been shown to have increased levels of neuropeptides, including a member of the VIP/glucagon/secretin or IL-2 family of peptides including peptide fragments and analogs. One possible explanation for this observation is that an endogenously expressed member of the VIP/glucagon/secretin or IL-2 family of peptides including peptide fragments and analogs may be biologically inactive (or partially inactivated). Because the circulating peptide is inactive, and its effects not realized, additional peptide is continually produced to achieve the desired effect. Administration of a member of the VIP/glucagon/secretin or IL-2 family of peptides including peptide fragments and analogs in a composition of the invention, which maintains a member of the VIP/glucagon/secretin or IL-2 family of peptides including peptide fragments and analogs in a biologically active conformation, would be expected to actuate biological processes dependent on a member of the VIP/glucagon/secretin or IL-2 family of peptides including peptide fragments and analogs that the endogenous inactive peptide cannot. As an alternative explanation, VP receptors are rendered dysfunctional to the extent that native VIP cannot interact, whereas VIP in a micelle composition of the invention is able to either recognize and interact with the modified receptor, or able to effect its biological activity through a non-receptor mediated pathway.

Sterically stabilized crystalline products of the invention are particularly useful for administration of anti-cancer agents. For example, crystalline products of the invention comprising paclitaxel (Taxol®) as the insoluble compound and VIP as the targeting agent can be targeted to breast cancer cells which are known to express higher levels of VIP receptor than normal breast cells, or express receptors with higher affinity of binding for VIP. Paclitaxel (Taxol®) has been shown to selectively kill breast cancer cells.

Cosmetic uses for the micelle and crystalline products of the invention include anti-aging , anti-wrinkling, and antioxidant activities, as well as use as a sunscreen.

The present invention is further illustrated by way of the following examples which used the following materials: lipids: L-α-egg yolk phosphatidylcholine type V-E in chloroform: methanol (9:1) (Lot #34H8395, and 75H8368), L-α-egg yolk phosphatidyl-D-α-Glycerol in chloroform: methanol (98:2) (Lot #72H8431, and 85H8395), and cholesterol (Lot #60H0476) from Sigma Chemical Co. (St. Louis, Mo.). Di-Palmitoyl-phosphatidyl choline (Lot #LP-04-01-112-187) from Sygenal Ltd. (Switzerland). PEG-DSPE in lyophilized powder form (Lot #180PHG2PK-26) from Avanti Polar Lipids Inc. (Alabaster, Ala.). Peptides: VIP (Lot #K02012A1, F02018A1, and K02018A1), VIP fragment 1-12 (Lot #H05009T1), VIP fragment 10-28 (Lot #NB0222), and Vasopressin (Lot #SD1051A) from American Peptide Co. (Sunnyvale, Calif.). Other bio-products: Bovine Brain Calmodulin (Lot #B10537) from Calbiochem Intl. (La Jolla, Calif.). ELISA assay kit (Lot #976605) from Peninsula Laboratories (Belmont, Calif.). Various chemicals: trehalose (Lot #43H7060), 2,4-diaminophenol (amidol, Lot #74H3652), ammonium molybdate (Lot #42H3506), sodium bisulfite (Lot #41H09432), HEPES (Lot #43H5720), and sodium chloride (Lot #22H0724) from Sigma Chemicals Co. (St. Louis, Mo.). Sodium dodecyl sulfate (Lot #11120KX) from Aldrich Chemical Co., Inc. Perchloric acid 70% (Lot #945567), chloroform HPLC grade (Lot #902521) and potassium phosphate monobasic (Lot #914723) from Fisher Sci. (Pittsburgh, Pa.).“Inflammation” as used herein refers to a localized, protective response elicited by injury or destruction of tissues, which serves to destroy, dilute or wall off (sequester) both the injurious agent and the injured tissue. Inflammation is notably associated with influx of leukocytes and or neutrophil chemotaxis. Inflammation may result from infection with pathogenic organisms and viruses and from noninfectious means such as trauma or reperfusion following myocardial infarction or stroke, immune response to foreign antigen, and autoimmune responses. Accordingly, inflammatory disorders amenable to the invention encompass disorders associated with reactions of the specific defense system as well as with reactions of the non-specific defense system.

As used herein, the term “specific defense system” refers to the component of the immune system that reacts to the presence of specific antigens. Examples of inflammation resulting from a response of the specific defense system include the classical response to foreign antigens, autoimmune diseases, and delayed type hypersensitivity response mediated by T-cells. Chronic inflammatory diseases, the rejection of solid transplanted tissue and organs, e.g., kidney and bone marrow transplants, and graft versus host disease (GVHD), are further examples of inflammatory reactions of the specific defense system.

The term “non-specific defense system” as used herein refers to inflammatory disorders that are mediated by leukocytes that are incapable of immunological memory (e.g., granulocytes, macrophages). Examples of inflammation that result, at least in part, from a reaction of the non-specific defense system include inflammation associated with conditions such as adult (acute) respiratory distress syndrome (ARDS) or multiple organ injury syndromes; reperfusion injury; acute glomerulonephritis; reactive arhritis; dermatoses with acute inflammatory components; acute purulent meningitis or other central nervous system inflammatory disorders such as stroke; thermal injury; inflammatory bowel disease; granulocyte transfusion associated syndromes; and cytokine-induced toxicity.

“Autoimmune disease” as used herein refers to any group of disorders in which tissue injury is associated with humoral or cell-mediated responses to the body's own constituents. “Allergic disease” as used herein refers to any symptoms, tissue damage, or loss of tissue function resulting from allergy. “Arthritic disease” as used herein refers to any disease that is characterized by inflammatory lesions of the joints attributable to a variety of etiologies. “Dermatitis” as used herein refers to any of a large family of diseases of the skin that are characterized by inflammation of the skin attributable to a variety of etiologies. “Transplant rejection” as used herein refers to any immune reaction directed against grafted tissue (including organs or cells (e.g., bone marrow), characterized by a loss of function of the grafted and surrounding tissues, pain, swelling, leukocytosis, and thrombocytopenia.

The therapeutic methods of the present invention include methods for the amelioration of disorders associated with inflammatory cell activation. “Inflammatory cell activation” refers to the induction by a stimulus (including, but not limited to, cytokines, antigens or auto-antibodies) of a proliferative cellular response, the production of soluble mediators (including but not limited to cytokines, oxygen radicals, enzymes, prostanoids, or vasoactive amines), or cell surface expression of new or increased numbers of mediators (including, but not limited to, major histocompatability antigens or cell adhesion molecules) in inflammatory cells (including but not limited to monocytes, macrophages, T lymphocytes, B lymphocytes, granulocytes (polymorphonuclear leukocytes including neutrophils, basophils, and eosinophils), mast cells, dendritic cells, Langerhans cells, and endothelial cells). It will be appreciated by persons skilled in the art that the activation of one or a combination of these phenotypes in these cells can contribute to the initiation, perpetuation, or exacerbation of an inflammatory disorder.

The present invention enables methods of treating various diseases associated with or characterized by inflammation, for example, arthritic diseases such as rheumatoid arthritis, osteoarthritis, gouty arthritis, spondylitis; Behcet disease; sepsis, septic shock, endotoxic shock, gram negative sepsis, gram positive sepsis, and toxic shock syndrome; multiple organ injury syndrome secondary to septicemia, trauma, or hemorrhage; ophthalmic disorders such as allergic conjunctivitis, vernal conjunctivitis, uveitis, and thyroid-associated ophthalmopathy; eosinophilic granuloma; pulmonary or respiratory disorders such as asthma, chronic bronchitis, allergic rhinitis, ARDS, chronic pulmonary inflammatory disease (e.g., chronic obstructive pulmonary disease), silicosis, pulmonary sarcoidosis, pleurisy, alveolitis, vasculitis, pneumonia, bronchiectasis, and pulmonary oxygen toxicity; reperfusion injury of the myocardium, brain, or extremities; fibrosis such as cystic fibrosis; keloid formation or scar tissue formation; atherosclerosis; autoimmune diseases such as systemic lupus erythematosus (SLE), autoimmune thyroiditis, multiple sclerosis, some forms of diabetes, and Reynaud's syndrome; transplant rejection disorders such as GVHD and allograft rejection; chronic glomerulonephritis; inflammatory bowel diseases such as Crohn's disease, ulcerative colitis and necrotizing enterocolitis; inflammatory dermatoses such as contact dermatitis, atopic dermatitis, psoriasis, or urticaria; fever and myalgias due to infection; central or peripheral nervous system inflammatory disorders such as meningitis, encephalitis, and brain or spinal cord injury due to minor trauma; Sjorgren's syndrome; diseases involving leukocyte diapedesis; alcoholic hepatitis; bacterial pneumonia; antigen-antibody complex mediated diseases; hypovolemic shock; Type I diabetes mellitus; acute and delayed hypersensitivity; disease states due to leukocyte dyscrasia and metastasis; thermal injury; granulocyte transfusion associated syndromes; and cytokine-induced toxicity.

Autoimmune disorders which may be treated using a protein of the present invention include, for example, connective tissue disease, multiple sclerosis, systemic lupus erythematosus, rheumatoid arthritis, autoimmune pulmonary inflammation, Guillain Barre syndrome, autoimmune thyroiditis, insulin dependent diabetes mellitis, myasthenia gravis, graft versus host disease and autoimmune inflammatory eye disease. Such a protein (or antagonists thereof, including antibodies) of the present invention may also to be useful in the treatment of allergic reactions and conditions (e.g., anaphylaxis, serum sickness, drug reactions, food allergies, insect venom allergies, mastocytosis, allergic rhinitis, hypersensitivity pneumonitis, urticaria, angioedema, eczema, atopic dermatitis, allergic contact dermatitis, erythema multiforme, Stevens Johnson syndrome, allergic conjunctivitis, atopic keratoconjunctivitis, venereal keratoconjunctivitis, giant papillary conjunctivitis and contact allergies), such as asthma (particularly allergic asthma) or other respiratory problems.

The present invention also provides methods of treating cancer in an animal, comprising administering to the animal an effective amount of a compound that inhibits DNA-PK activity. The invention is further directed to methods of inhibiting cancer cell growth, including processes of cellular proliferation, invasiveness, and metastasis in biological systems. Methods include use of a compound of the invention as an inhibitor of cancer cell growth. Preferably, the methods are employed to inhibit or reduce cancer cell growth, invasiveness, metastasis, or tumor incidence in living animals, such as mammals. Methods of the invention are also readily adaptable for use in assay systems, e.g., assaying cancer cell growth and properties thereof, as well as identifying compounds that affect cancer cell growth.

Compounds of the invention are possess one or more desirable but unexpected combinations of properties, including increased activity and/or solubility, and reduction of negative side effects. These compounds have been found to inhibit cancer growth, including proliferation, invasiveness, and metastasis, thereby rendering them particularly desirable for the treatment of cancer. In particular, compounds of the invention exhibit cancer-inhibitory properties at concentrations that appear to be substantially free of side effects. These compounds are therefore useful for extended treatment protocols, where the use of conventional chemotherapeutic compounds can exhibit undesirable side effects. For example, the coadministration of a compound of the invention with another, more toxic, chemotherapeutic agent can achieve beneficial inhibition of a cancer, while effectively reducing the toxic side effects in the patient.

In addition, the properties of hydrophilicity and hydrophobicity of the compounds of the invention are well balanced, thereby enhancing their utility for both in vitro and especially in vivo uses, while other compounds lacking such balance are of substantially less utility. Specifically, compounds of the invention have an appropriate degree of solubility in aqueous media which permits absorption and bioavailability in the body, while also having a degree of solubility in lipids which permits the compounds to traverse the cell membrane to a putative site of action. Thus, compounds of the invention are maximally effective when they can be delivered to the site of the tumor and they enter the tumor cells.

The cancers treatable by methods of the present invention preferably occur in mammals. Mammals include, for example, humans and other primates, as well as pet or companion animals such as dogs and cats, laboratory animals such as rats, mice and rabbits, and farm animals such as horses, pigs, sheep, and cattle.

Tumors or neoplasms include growths of tissue cells in which the multiplication of the cells is uncontrolled and progressive. Some such growths are benign, but others are termed “malignant” and may lead to death of the organism. Malignant neoplasms or “cancers” are distinguished from benign growths in that, in addition to exhibiting aggressive cellular proliferation, they may invade surrounding tissues and metastasize. Moreover, malignant neoplasms are characterized in that they show a greater loss of differentiation (greater “dedifferentiation”), and of their organization relative to one another and their surrounding tissues. This property is also called “anaplasia.”

Neoplasms treatable by the present invention also include solid tumors, i.e., carcinomas and sarcomas. Carcinomas include those malignant neoplasms derived from epithelial cells which infiltrate (invade) the surrounding tissues and give rise to metastases. Adenocarcinomas are carcinomas derived from glandular tissue, or which form recognizable glandular structures. Another broad category or cancers includes sarcomas, which are tumors whose cells are embedded in a fibrillar or homogeneous substance like embryonic connective tissue. The invention also enables treatment of cancers of the myeloid or lymphoid systems, including leukemias, lymphomas and other cancers that typically do not present as a tumor mass, but are distributed in the vascular or lymphoreticular systems.

The type of cancer or tumor cells amenable to treatment according to the invention include, for example, ACTH-producing tumor, acute lymphocytic leukemia, acute nonlymphocytic leukemia, cancer of the adrenal cortex, bladder cancer, brain cancer, breast cancer, cervical cancer, chronic lymphocytic leukemia, chronic myelocytic leukemia, colorectal cancer, cutaneous T-cell lymphoma, endometrial cancer, esophageal cancer, Ewing's sarcoma, gallbladder cancer, hairy cell leukemia, head and neck cancer, Hodgkin's lymphoma, Kaposi's sarcoma, kidney cancer, liver cancer, lung cancer (small and non-small cell), malignant peritoneal effusion, malignant pleural effusion, melanoma, mesothelioma, multiple myeloma, neuroblastoma, glioma, non-Hodgkin's lymphoma, osteosarcoma, ovarian cancer, ovarian (germ cell) cancer, pancreatic cancer, penile cancer, prostate cancer, retinoblastoma, skin cancer, soft tissue sarcoma, squamous cell carcinomas, stomach cancer, testicular cancer, thyroid cancer, trophoblastic neoplasms, uterine cancer, vaginal cancer, cancer of the vulva, and Wilms's tumor.

The invention is particularly illustrated herein in reference to treatment of certain types of experimentally defined cancers. In these illustrative treatments, standard state-of-the-art in vitro and in vivo models have been used. These methods can be used to identify agents that can be expected to be efficacious in in vivo treatment regimens. However, it will be understood that the method of the invention is not limited to the treatment of these tumor types, but extends to any solid tumor derived from any organ system. Cancers whose invasiveness or metastasis is associated with DNA-PK expression or activity are especially susceptible to being inhibited or even induced to regress by means of the invention.

The invention further relates to radiosensitizing tumor cells. The term “radiosensitizer,” as used herein, is defined as a molecule, preferably a low molecular weight molecule, administered to animals in therapeutically effective amounts to increase the sensitivity of the cells to be radiosensitized to electromagnetic radiation and/or to promote the treatment of diseases that are treatable with electromagnetic radiation. Diseases that are treatable with electromagnetic radiation include neoplastic diseases, benign and malignant tumors, and cancerous cells.

Electromagnetic radiation treatment of other diseases not listed herein is also contemplated by the present invention. The terms “electromagnetic radiation” and “radiation” as used herein include, but are not limited to, radiation having the wavelength of 10-20 to 100 meters. Preferred embodiments of the present invention employ the electromagnetic radiation of: garnma-radiation (10-20 to 10-13 m), X-ray radiation (10-12 to 10-9 m), ultraviolet light (10 nm to 400 nm), visible light (400 nm to 700 nm), infrared radiation (700 nm to 1.0 mm), and microwave radiation (1 mm to 30 cm).

Radiosensitizers are known to increase the sensitivity of cancerous cells to the toxic effects of electromagnetic radiation. Several mechanisms for the mode of action of radiosensitizers have been suggested in the literature including: hypoxic cell radiosensitizers, e.g., 2-nitroimidazole compounds, and benzotriazine dioxide compounds) promote the reoxygenation of hypoxic tissue and/or catalyze the generation of damaging oxygen radicals; non-hypoxic cell radiosensitizers (e.g., halogenated pyrimidines) can be analogs of DNA bases and preferentially incorporate into the DNA of cancer cells and thereby promote the radiation ion-induced breaking of DNA molecules and/or prevent the normal DNA repair mechanisms; and various other potential mechanisms of action have been hypothesized for radiosensitizers in the treatment of disease.

Many cancer treatment protocols currently employ radiosensitizers activated by the electromagnetic radiation of X-rays. Examples of X-ray activated radiosensitizers include, but are not limited to, the following: metronidazole, misonidazole, desmethylmisonidazole, pimonidazole, etanidazole, nimorazole, mitomycin C, RSU 1069, SR 4233, E09, RB 6145, nicotinamide, 5-bromodeoxyuridine (BUdR), 5-iododeoxyuridine (IUdR), bromodeoxycytidine, fluorodeoxyuridine (FUdR), hydroxyurea, cisplatin, and therapeutically effective analogs and derivatives of the same.

Photodynamic therapy (PDT) of cancers employs visible light as the radiation activator of the sensitizing agent. Examples of photodynamic radiosensitizers include the following, but are not limited to: hematoporphyrin derivatives, Photofrin(r), benzoporphyrin derivatives, NPe6, tin etioporphyrin (SnET2), pheoborbide-a, bacteriochlorophyll-a, naphthalocyanines, phthalocyanines, zinc phthalocyanine, and therapeutically effective analogs and derivatives of the same.

Radiosensitizers may be administered in conjunction with a therapeutically effective amount of one or more other compounds, including but not limited to: compounds that promote the incorporation of radiosensitizers to the target cells; compounds that control the flow of therapeutics, nutrients, and/or oxygen to the target cells; chemotherapeutic agents that act on the tumor with or without additional radiation; or other therapeutically effective compounds for treating cancer or other disease. Examples of additional therapeutic agents that may be used in conjunction with radiosensitizers include, but are not limited to: 5-fluorouracil (5-FU), leucovorin, 5(-amino-5(-deoxythymidine, oxygen, carbogen, red cell transfusions, perfluorocarbons (e.g., Fluosol(r)-DA), 2,3-DPG, BW12C, calcium channel blockers, pentoxyfylline, anti-angiogenesis compounds, hydralazine, and L-BSO. Examples of chemotherapeutic agents that may be used in conjunction with radiosensitizers include, but are not limited to: adriamycin, camptothecin, carboplatin, cisplatin, daunorubicin, doxorubicin, interferon (alpha, beta, gamma), interleukin 2, irinotecan, docetaxel, paclitaxel, topotecan, and therapeutically effective analogs and derivatives of the same.

The invention can also be practiced by including with a compound of the invention another anti-cancer chemotherapeutic agent, such as any conventional chemotherapeutic agent. The combination of the tetracycline compound with such other agents can potentiate the chemotherapeutic protocol. Numerous chemotherapeutic protocols will present themselves in the mind of the skilled practitioner as being capable of incorporation into the method of the invention. Any chemotherapeutic agent can be used, including alkylating agents, antimetabolites, hormones and antagonists, radioisotopes, as well as natural products. For example, the compound of the invention can be administered with antibiotics such as doxorubicin and other anthracycline analogs, nitrogen mustards such as cyclophosphamide, pyrimidine analogs such as 5-fluorouracil, cisplatin, hydroxyurea, paclitaxel (Taxol®) and its natural and synthetic derivatives, and the like. As another example, in the case of mixed tumors, such as adenocarcinoma of the breast, where the tumors include gonadotropin-dependent and gonadotropin-independent cells, the compound can be administered in conjunction with leuprolide or goserelin (synthetic peptide analogs of LH-RH). Other antineoplastic protocols include the use of a tetracycline compound with another treatment modality, e.g., surgery, radiation, etc., also referred to herein as “adjunct antineoplastic modalities.” Thus, the method of the invention can be employed with such conventional regimens with the benefit of reducing side effects and enhancing efficacy.

Therapeutic compositions are within the scope of the present invention. Such pharmaceutical compositions may comprise a therapeutically effective amount of a micelle composition alone or in admixture with a pharmaceutically or physiologically acceptable formulation agent selected for suitability with the mode of administration. Pharmaceutical compositions may comprise a therapeutically effective amount of one or more micelle compositions in admixture with a pharmaceutically or physiologically acceptable formulation agent selected for suitability with the mode of administration.

The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen sulfite); buffers (such as borate, bicarbonate, Tris HCl, citrates, phosphates, other organic acids); bulking agents (such as mannitol or glycine), chelating agents (such as ethylenediamine tetraacetic acid (EDTA)); complexing agents (such as caffeine, polyvinylpyrrolidone, beta cyclodextrin or hydroxypropyl beta cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company, 1990).

The pharmaceutical micelle compositions can be selected for parenteral delivery. Alternatively, the compositions may be selected for inhalation or for delivery through the digestive tract, such as orally. The preparation of such pharmaceutically acceptable compositions is within the skill of the art.

In one embodiment, a pharmaceutical composition may be formulated for inhalation. For example, a micelle composition may be formulated as a dry powder for inhalation. Pharmaceutical micelle composition inhalation solutions may also be formulated with a propellant for aerosol delivery. In yet another embodiment, solutions may be nebulized. Pulmonary administration is further described in PCT Application No. PCT/US94/001875, which describes pulmonary delivery of chemically modified proteins.

It is also contemplated that certain formulations may be administered orally. In one embodiment of the present invention, micelle compositions which are administered in this fashion can be formulated with or without those carriers customarily used in the compounding of solid dosage forms such as tablets and capsules. For example, a capsule may be designed to release the active portion of the formulation at the point in the gastrointestinal tract when bioavailability is maximized and pre systemic degradation is minimized. Additional agents can be included to facilitate absorption of the micelle composition. Diluents, flavorings, low melting point waxes, vegetable oils, lubricants, suspending agents, tablet disintegrating agents, and binders may also be employed.

Another pharmaceutical composition may involve an effective quantity of micelle compositions in a mixture with non toxic excipients which are suitable for the manufacture of tablets. By dissolving the tablets in sterile water, or other appropriate vehicle, solutions can be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical micelle compositions will be evident to those skilled in the art, including formulations involving micelle compositions in sustained or controlled delivery formulations. Techniques for formulating a variety of other sustained or controlled delivery means, such as liposome carriers, bio erodible microparticles or porous beads and depot injections, are also known to those skilled in the art.

The pharmaceutical micelle composition to be used for in vivo administration typically must be sterile. This may be accomplished by filtration through sterile filtration membranes. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or a dehydrated or lyophilized powder. Such formulations may be stored either in a ready to use form or in a form (e.g., lyophilized) requiring reconstitution prior to administration.

An effective amount of a pharmaceutical micelle composition to be employed therapeutically will depend, for example, upon the therapeutic context and objectives. One skilled in the art will appreciate that the appropriate dosage levels for treatment will thus vary depending, in part, upon the molecule delivered, the indication for which the micelle composition is being used, the route of administration, and the size (body weight, body surface or organ size) and condition (the age and general health) of the patient. Accordingly, the clinician may titer the dosage and modify the route of administration to obtain the optimal therapeutic effect. A typical dosage may range from about 0.1 mg/kg to up to about 100 mg/kg or more, depending on the factors mentioned above. In other embodiments, the dosage may range from 0.1 mg/kg up to about 100 mg/kg; or 1 mg/kg up to about 100 mg/kg; or 5 mg/kg up to about 100 mg/kg.

The frequency of dosing will depend upon the pharmacokinetic parameters of the micelle composition in the formulation used. Typically, a clinician will administer the composition until a dosage is reached that achieves the desired effect. The composition may therefore be administered as a single dose, or as two or more doses (which may or may not contain the same amount of the desired molecule) over time, or as a continuous infusion via implantation device or catheter. Further refinement of the appropriate dosage is routinely made by those of ordinary skill in the art and is within the ambit of tasks routinely performed by them. Appropriate dosages may be ascertained through use of appropriate dose response data.

The route of administration of the pharmaceutical composition is in accord with known methods, e.g. orally, through injection by intravenous, intraperitoneal, intracerebral (intra parenchymal), intracerebroventricular, intramuscular, intra ocular, intraarterial, intraportal, or intralesional routes, by sustained release systems or by implantation devices. Where desired, the compositions may be administered by bolus injection or continuously by infusion, or by implantation device.

Alternatively or additionally, the composition may be administered locally via implantation of a membrane, sponge, or another appropriate material on to which the desired molecule has been absorbed or encapsulated. Where an implantation device is used, the device may be implanted into any suitable tissue or organ, and delivery of the desired molecule may be via diffusion, timed release bolus, or continuous administration.

In some cases, it may be desirable to use pharmaceutical micelle compositions in an ex vivo manner. In such instances, cells, tissues, or organs that have been removed from the patient are exposed to pharmaceutical micelle compositions after which the cells, tissues and/or organs are subsequently implanted back into the patient.

In addition to the modes of administration enclosed herein, compositions of the invention can be introduced for treatment into a mammal by other modes, such as but not limited to, intra-articular, intra-tumor, cerebrospinal, intra-arterial, intra-peritoneal, intra-rectal and colon, intra-lesion, topical, subconjunctival, intra-bladder, intra-vaginal, epidural, intracostal, intra-dermal, inhalation, transdermal; trans-serosal, intra-buccal, oral, intra-nasal, intra-muscular, dissolution in the mouth or other body cavities, instillation to the airway, insuflation through the airway, injection into vessels, tumors, organ and the like, and injection or deposition into cavities in the body of a mammal.

In addition to the treatment of other diseases or disorders disclosed herein, compositions of the invention can be used for the treatment of cerebrovascular ischemia, erectile dysfunction, female sexual arousal dysfunction, motor neuron disease, neuropathy, pain, depression, anxiety disorders, brain trauma, sepsis, septic shock, shock, adult respiratory distress syndrome, meconium aspiration, infantile respiratory distress syndrome, memory impairments, dementia, cognitive disorder, autism, central nervous system disease (such as Parkinson's disease, Alzheimer's disease), migraine, cerebral palsy, neurodegenerative diseases, stroke, hypertension, pulmonary hypertension, portal hypertension, ischemic heart disease, arthritis, osteoartritis, gouty arthritis, crystal-induced arthritis, snoring, arteritis, rhinitis, psoriasis, radiation-induced tissue injury, septicemia, exocrine pancreatic insufficiency, pancreatitis, spondyloarthropathies, hypersensitivity, anaphylaxis, encephalopathy, vascular insufficiency, tetanus, tenosynovitis, synovitis, ischemia, neuritis, nerve palsy, pressure ulcers, progressive multifocal leukoencephalopathy, meningitis, pericarditis, myocarditis, inflammation, multiple sclerosis, multiple organ system failure, nephritis, obliterative bronchiolitis, bronchiolitis obliterans-organizing pneumonia, encephalitis, diversion colitis and pouchitis, inflammatory polyps, polymyositis, polychondritis, polyarthritis, pemphigus, bullous pemphigoid, acne, rosacea, nephritis, glomerulonephritis, cancer, interstitial lung disease, idiopathic pulmonary fibrosis, sarcoidosis, tuberous sclerosis, vasculitis, toxic shock syndrome, asthma, chroic obstructive pulmonary disease, bronchitis, emphysema, bronchiectasis, acute coronary syndrome, angina pectoris, gastroparesis, mental retardation, rheumatioid arthritis, inflammatory bowel disease, Crohn's disease, ulcerative colitis, muscle disease, autoimmune diseases (such as lupus erythematosus, scleroderma, dermatomyosytis, Sjogren's syndrome, CREST), Raynaud's phenomenon, Bierger's disease, peripheral vascular disease, chronic venous ulcers, dernmatitis, diabetes mellitus, atherosclerosis, myocardial infarction, gastric and duodenal ulcers, ischemic heart disease, fibrosis, restenosis, thrombosis, caridiac failure, cardiomyopathy, encephalopathy, cerebritis, ankylosing spondylitis, osteoarthritis, renal failure, neuritis, neuropathy, spondylosis, retinal disease, prevention of neuronal cell death in a mammal, food impaction, VIPoma, wound healing, constipation, arthropathy, pre-eclampsia, burns, skin ulcers, toxic megacolon, organ, tissue and cell preservation, Reiter's syndrome, psoriatic arthritis, prevention of hypotension and hypertension evoked by said peptides.

The following examples are intended for illustration purposes only, and should not be construed as limiting the scope of the invention in any way.

### EXAMPLE 1

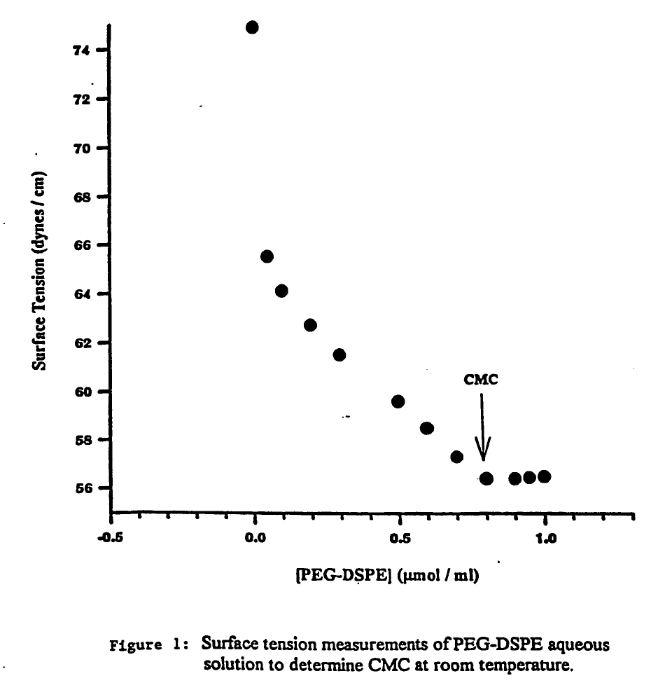

According to this example, VIP was incorporated into sterically stabilized micelles according to the following procedure. In order to determine the concentration of PEG-DSPE needed to prepare micelles, surface tension studies of PEG-DSPE aqueous solutions were performed. The critical micellar concentration was found to be 0.5 to 1.0 μM, thus 1.0 μM of PEG-DSPE was used to ensure formation of micelles (FIG. 1). PEG-DSPE lipid (1 μmol/ml) was dissolved in chloroform and mixed in a round bottom flask. The organic solvent was evaporated using a rotoevaporater at a bath water temperature of 45° C. (Labconco, Kansas City, Mo.). Complete dryness was achieved by desiccation under vacuum overnight. The dry lipid film was hydrated with saline (0.15 N, pH 6.8) or HEPES buffer (10 mM, pH 7.4). The solution was incubated with human VIP (13 μg/ml) for 30 min before use in circular dichroism. Human VIP (0.1 nmol/ml) was added to the phospholipid micelle suspension and incubated for 2 hours at room temperature before use in cheek pouch studies.

### EXAMPLE 2

According to this example, sterically stabilized micelles comprising VIP and calmodulin were prepared according to the procedure of Example 1 wherein the method of that example was followed to prepare the SSM suspension and during the incubation stage 100 μl of 10⁻⁹ M CaM was added to 900 μl of VIP-micelles (giving a total CaM concentration of 10⁻¹⁰ M) and incubated for 2 h at 4° C. before use in circular dichroism. Human VIP (0.1 nmol/ml) and 100 μl of 10⁻⁹ M CaM was added to 900 μl of phospholipid micelles (giving a total CaM concentration of 10⁻¹⁰ M) and incubated for 2 hours at room temperature before use in cheek pouch studies. VIP concentration of 0.1 nmol was used to allow comparison of results with VIP in sterically stabilized micelle formulation.

### EXAMPLE 3

According to this example, the size of the vesicles was determined by quasi elastic light scattering (NICOMP model 270 submicron particle sizer, Pacific Scientific, Menlo Park, Calif.). This device contains a 5 mW Helium-Neon Laser at an excitation wavelength of 623.8 nm and with a 64-channel autocorrelation function, a temperature-controlled scattering cell holder and an ADM 11 video display terminal computer (learr Siegler Inc.) for analyzing the fluctuations in scattered light intensity generated by the diffusion of particles in solution. The mean hydrodynamic particle diameter, dₕ, was obtained from the Stokes-Einstein relation using the measured diffusion coefficient obtained from analysis of autocorrelation functions accumulated, for 30 min. The following instrument settings were used; temperature, 23° C.; viscosity, 0.9325 cp; refractive index, 1.333; and scattering angle, 90°. The sterically stabilized phospholipid micelles (SSM) loaded with vasoactive intestinal peptide (VIP) had a final mean size of ˜17.9±0.6 nm.

### EXAMPLE 4

According to this example, circular dichroism (CD) experiments were performed to determine the conformation changes of VIP in phospholipid micelles, pH and temperature changes, and in aqueous solutions. CD spectra were recorded on a JASCO J-700 spectropolarimeter using a fused quartz cell of 1 cm pathlength. Spectra in 0.15 N saline (pH 6.8) and 5 mM Hepes buffer (pH 7.4) were measured at a peptide concentration of 4 μM and at a lipid concentration of 1 mM. The effects of CaM, pH (6.8 or 7.4) and temperature (25° C. or 37° C.) on VIP conformation were also studied. The conformation of VIP fragments was likewise studied. All measurements were carried out at room temperature (˜25° C.) unless otherwise noted. A bandwidth of 1.0 nm and a step resolution of 0.5 nm were used to collect an average of 9 accumulations/sample at the near-UV range (200-260 nm wavelength). The peptide spectra shown have the background buffer scans, and empty vesicle scans subtracted and were smoothed using the noise reduction function. The temperature during spectral analysis was maintained by using a circulating water bath attached to a jacket surrounding the fused quartz CD cell. The percent helical characterization of VIP was determined by a method of Haghjoo et al., Peptide Research 9 (6):327-331 (1996): % helicity=[−(θ₂₀₈+4000)/29 000]*100 and are reported in table 1.

TABLE 1
Percent helical characteristic of the peptide obtained by
deconvolution using CD analysis.

| | % Helicity | | |
|---|---|---|---|
| | (in the presence of) | | Molar Ratio |
| Peptide | Saline | Phospholipids | Peptide:Phospholipid |
| VIP-DPPC*/PG | 2 | 2 | 0.0057 |
| VIP @ Room Temp. | 5 | 27 | 0.0057 |
| VIP @ 37° C. | 2 | 67 | 0.0057 |
| VIP + CaM | 1 | 42 | — |
| CaM | 0 | 0 | 0.0057 |
| VIP₁₋₁₂ | 0 | 0.05 | 0.0057 |
| VIP₁₀₋₂₈ | 3 | 18 | 0.0057 |
| Vasopressin | 8 | 16 | 0.0057 |

*Di-palmitoyl-phosphatidylcholine

According to the example, CD was used to determine the conformation of VIP in saline, Hepes buffer and phospholipid micelles at room temperature and at 37° C. The CD spectra analysis was performed after 13 μg of human VIP incubated with 1 ml PEG-DSPE (1 μmol) micelles for 30 min at room temperature as determined by preliminary studies. A bandwidth of 1.0 nm and a step resolution of 0.5 nm were used to collect an average of 9 accumulations/sample at near UV range (200-260 nm). The temperature was maintained during spectral analysis by a circulating water bath attached to a jacket surrounding the fused quartz CD cell. The evaluation of VIP molecule conformation in SSM by using circular dichroism was successful because the distortion caused by spherical particles was eliminate due to the small size and univesicular structure of the SSM. The dynamic nature of the micelles also enhanced the VIP interactions with phospholipids. The phospholipid micelles were ideal in our study of VIP conformation since it provided a hydrophobic core similar to the phospholipid bilayer of the SSL. Moreover, both the negative charge, and the hydrophilic layer provided by the PEG mimic the conditions of our SSL and make it possible to infer the VIP conformational results.

The spectral characteristics of VIP in pure water has been shown to be random coil but in organic solvents VIP has been shown to have an α-helix formation (Fournier et al., Peptides 5:160-177 (1984); Fry et al., Biochemistry 28:2399-2409 (1989); Theriault et al., Biopolymers 31:459-464 (1991)). Furthermore, short peptides capable of forming amphipathic helices are known to bind and penetrate lipid bilayers (Noda et al., Biochim. Biophys. Acta. 1191:324-330 (1994)). Based on these information VIP is expected to form a helical structure when associated with the micelle.

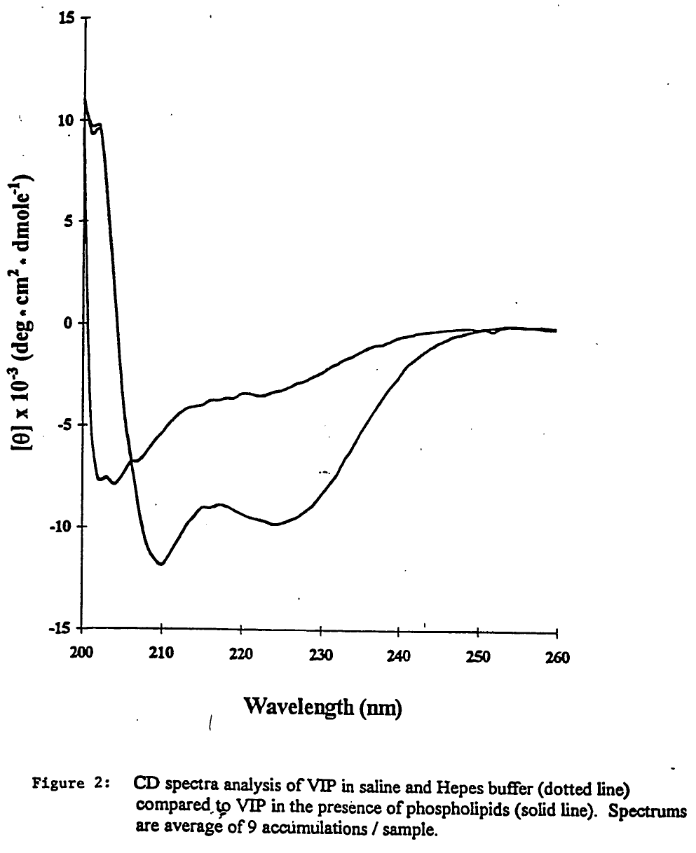

CD spectra of human VIP in saline (pH 6.8) and in the presence of phospholipid micelles are shown in FIG. 2. The studies showed that the peptide has a marked conformational sensitivity to its environment. In the saline, VIP, exhibited a minimum at 203 nm, showing that it is primarily a random coil structure. In the presence of phospholipid micelles, it had a double minimum at 208 nm and 222 nm characteristic of a predominantly α-helix conformation (Table 1).

CD spectra of human VIP in Hepes buffer (pH 7.4) and in the presence of phospholipid micelles are also shown in FIG. 2. (CD spectra analysis of VIP in saline and Hepes buffer (dotted line) compared to VIP in the presence of phospholipids (solid line). Spectrums are average of 9 accumulations/sample.) These studies showed that the peptide in Hepes buffer exhibited a minimum at 203 nm, showing that it is primarily a random coil structure. In the presence of phospholipid micelles, it had a double minimum at 208 nm and 222 nm characteristic of a predominantly α-helix conformation (Table 1). These results are similar to VIP in saline (pH 6.8).

Study of the CD spectra of vasoactive intestinal peptide in saline solution and in the presence of SSM have shown that VIP is in a mostly random coil configuration in saline but in a predominantly α-helix conformation in the presence of SSM. This indicates that VIP, in part, does indeed enter the hydrophobic core despite potential steric hindrance from PEG, and change to its more stable α-helix conformation. Unlike, many amphiphilic molecules the change in the pH showed no significant alterations in VIP conformation suggesting that VIP is not effected by the ionic environment in the range studied.

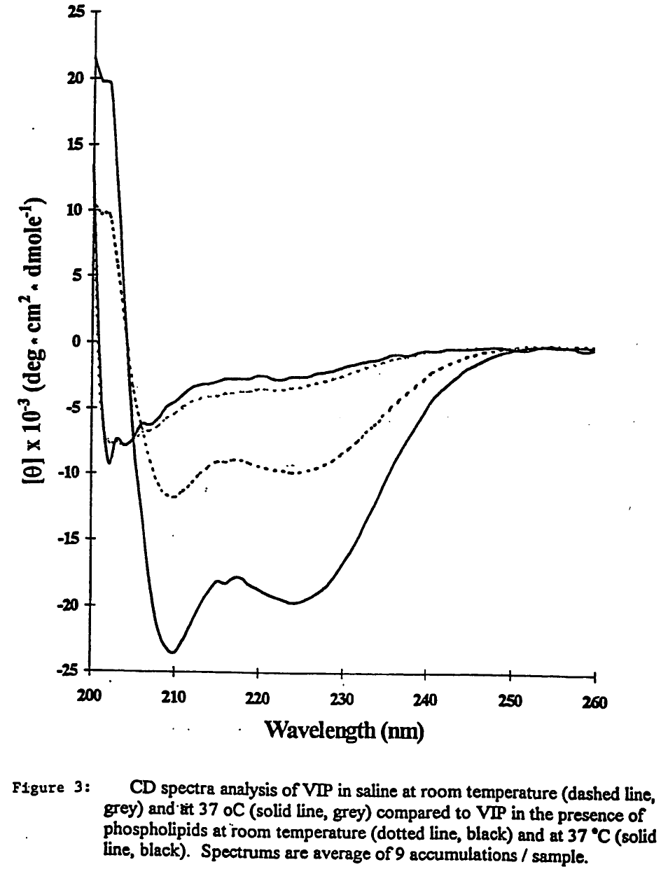

CD spectra of human VIP in saline and in the presence of phospholipid micelles at 37° C. are shown in FIG. 3. (CD spectra analysis of VIP in saline at room temperature (dashed line, grey) and at 37° C. (solid line, grey) compared to VIP in the presence of phospholipids at room temperature (dotted line, black) and at 37° C. (solid line, black). Spectrums are average of 9 accumulations/sample.) The studies showed that the absorbance intensity of the peptide in micelles increased at 37° C. compared to room temperature with no change in the spectral shape. The increase in absorbance intensity indicates an amplification of α-helix conformation, as shown by the tripling of the % helical content of VIP (Table 1).

This effect of temperature increase on VIP conformation in SSM is most likely due to the critical micellar temperature (CMT) effects, where CMT is the temperature at which micelles are formed. This increase in the temperature has been shown to be accompanied by an increase in the number of micelles (Nivaggioli et al., Langmuir. 11 (3):730-737 (1995)). Increase in the number of micelles leads to an increase in the hydrophobicity of the micellar suspension. Thus, an amplification of α-helix structure of VIP molecules is seen because more of the VIP molecules interact with the micelle or micellar core.

### EXAMPLE 5

According to this example, the method of example 4 was repeated to determine the conformation of VIP in saline and phospholipid micelles plus calmodulin (CaM). CD spectra measurements were performed after 13 μg/ml of VIP was incubated with 1.0 μmol/ml of phospholipid micelles for 30 min followed by 10⁻¹⁰ M CaM incubated with VIP in phospholipid micelles for 2 h at 4° C. (conjugation paper). A bandwidth of 1.0 nm and a step resolution of 0.5 nm were used to collect the average of 9 accumulations/sample at near UV range (200-260 nm).

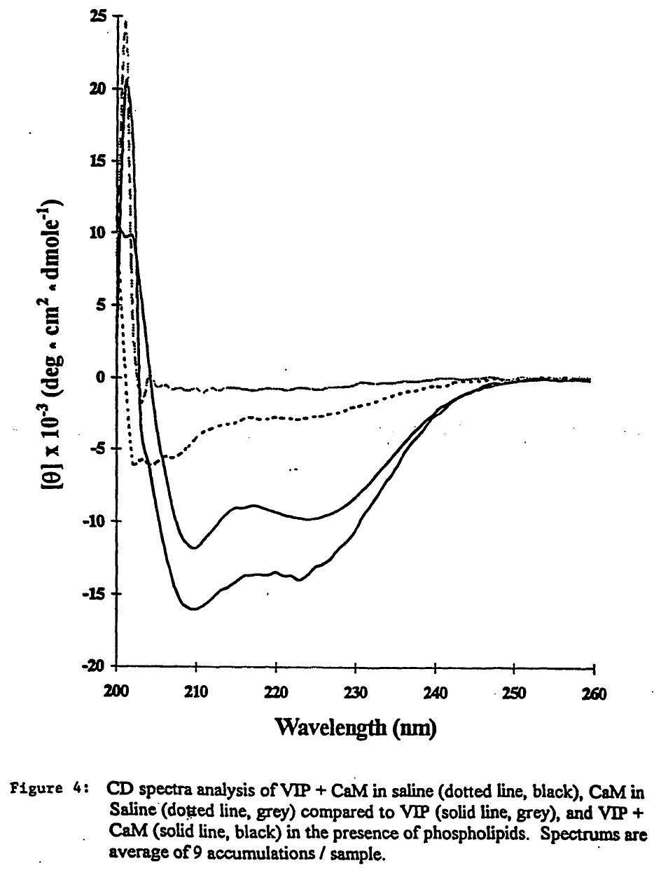

CD spectra of human VIP in saline and in the presence of phospholipid in micelles plus CaM are shown in FIG. 4. (CD spectra analysis of VIP+CaM in saline (dotted line, black), CaM in Saline (dotted line, grey) compared to VIP (solid line, grey), and VIP+CaM (solid line, black) in the presence of phospholipids. Spectrums are average of 9 accumulations/sample.) The studies showed that CaM increases the absorbance intensity of VIP in phospholipid micelles without changing the spectral shape. This increase in absorbance indicates an amplification of α-helix conformation, as seen by the doubling of the % helical content of VIP (Table 1). CaM alone had no significant effects on VIP conformation in saline (FIG. 4).

CaM seems to elicit an amplification of the α-helix structure of the VIP in the presence of phospholipids. CaM has been known to interact with phospholipids (Chiba et al., Life Sciences 47:953-960 (1990); Houbre et al., J. Biol. Chem. 266(11):71217131 (1991); Stallwood et al., J. Biol. Chem. 267:19617-19621(1992); Bolin, Neurochem. Int. 23:197-214 (1993); Paul et al., Neurochem. Int. 23:197-214 (1993)) and this interaction most likely expose the hydrophobic regions of CaM which induce an increase in the α-helix structure of the VIP. Furthermore, the addition of CaM may decrease the CMC causing the increase in micelle number, which further increases the hydrophobicity of the solution and leads to an amplification of α-helix structure.

### EXAMPLE 6

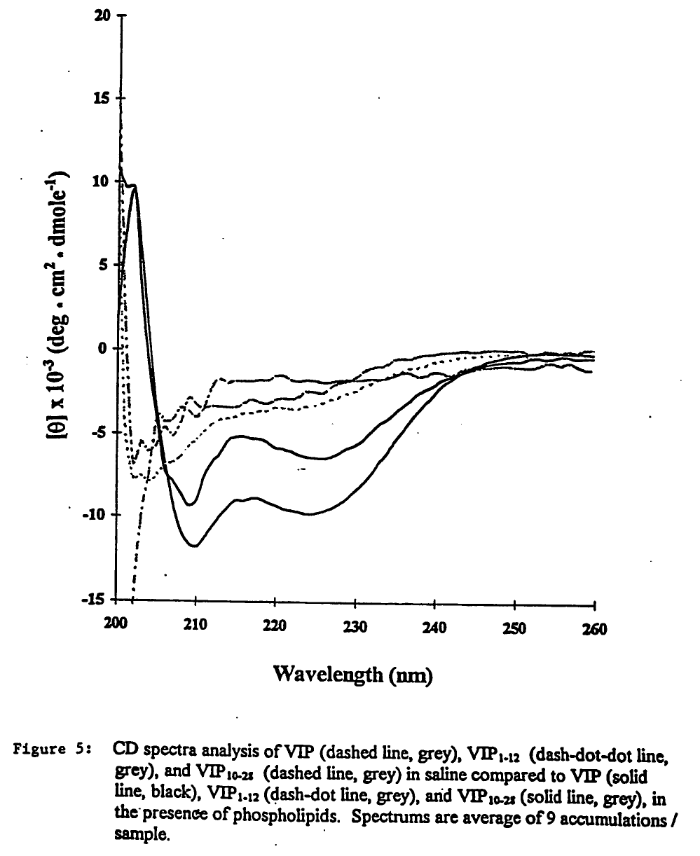

According to this example the method of example 4 was repeated to determine the conformation of VIP fragments in saline and phospholipid micelles. CD spectra measurements were performed after incubation of VIP fragments with phospholipid micelles, at a molar concentration equivalent to VIP (i.e. 9 μg/ml for VIP₁₀₋₂₈ fragment and 6 μg/ml for VIP₁₋₁₂ fragment), for 30 min. A bandwidth of 1.0 nm and a step resolution of 0.5 nm were used to collect the average of 9 accumulations/sample at near UV range (200-260 nm). Specifically, CD spectra of human VIP fragment (1-12) and (10-28) in the presence of phospholipid micelles are shown in FIG. 5. (CD spectra analysis of VIP (dashed line, grey), VIP₁₋₁₂ (dash-dot-dot line, grey), and VIP₁₀₋₂₈ (dashed line, grey) in saline compared to VIP (solid line, black), VIP₁₋₁₂ (dash-dot line, grey), and VIP₁₀₋₂₈ (solid line, grey), in the presence of phospholipids. Spectrums are average of 9 accumulations/sample.) The spectrum of VIP₁₋₁₂ fragment has a minima at 203 nm in saline and in the presence of SSM indicating a primarily random coil structure. Conversely, the spectrum of VIP₁₀₋₂₈ in the presence of SSM has a double minima at 208 nm and 225 nm suggesting a predominantly α-helix structure. The spectrum of VIP₁₀₋₂₈ in saline has a minima at 203 nm indicating a primarily random coil conformation. These effects correlate well with the % helicity of VIP fragments determined in saline and in the presence of phospholipids (Table 1).

The CD spectra of VIP fragments clearly indicate that the α-helix region of the peptide lies in the 10-28 amino acid sequence of the VIP. Others have also observed this phenomenon using CD spectra of VIP in organic solvents. The α-helix formation in VIP₁₀₋₂₈ aids in explaining its antagonistic bioactivity in vivo. Previously in the inventor's laboratory it was shown that VIP₁₀₋₂₈ fragment completely abolished native VIP response and attenuated VIP in SSL response in the hamster cheek pouch microcirculation (Séjourné et al., Pharm. Res. 14(3):362-365 (1997). This mechanism can be explained by the α-helix structure of VIP₁₀₋₂₈, which allows the fragment to bind the VIP-receptor site blocking the receptor interaction with VIP. VIP₁₀₋₂₈ has been reported to bind one type of VIP receptors on smooth muscles (Rorstad et al., Mol. Pharmacol. 37:971-977 (1990)).

### EXAMPLE 7

The method of example 4 was also repeated to determine the conformation of Vasopressin (VP) in saline and phospholipid micelles at room temperature and at 37° C. CD spectra measurements were performed after incubation of VP with phospholipid micelles, at a molar concentration equivalent to VIP (i.e. 4 μg/ml of VP in 1.0 μmol/ml phospholipids), for 30 min. A bandwidth of 1.0 nm and a step resolution of 0.5 nm were used to collect the average of 9 accumulations/sample at near UV range (200-260 nm). The temperature during spectral analysis was maintained by using a circulating water bath attached to a jacket surrounding the fused quartz CD cell.

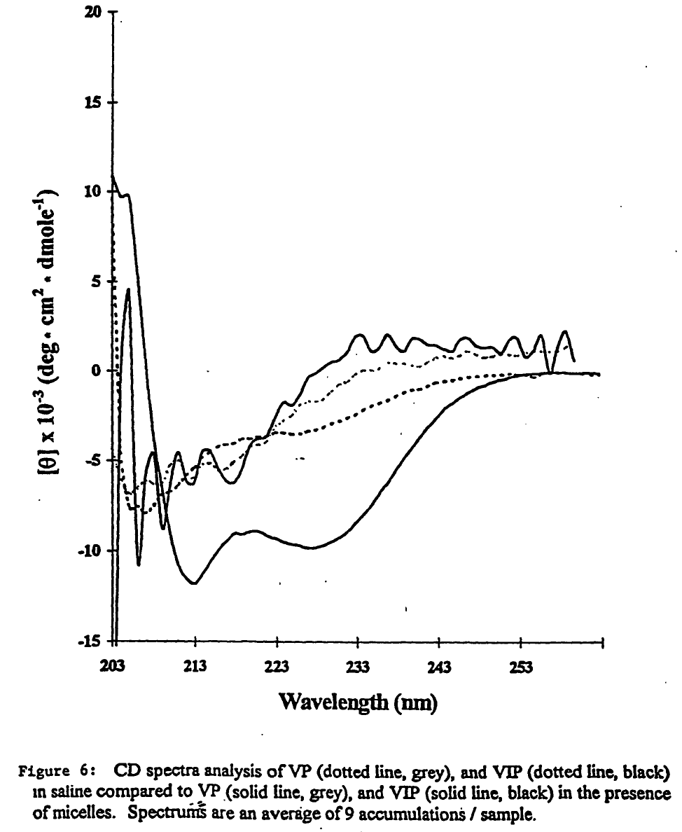

Vasopressin (VP) has been tested for long circulation hours and activity after administered as SSL. Therefore, in this study it was attempted to determine if VP also acts by association with the liposomal bilayer. VP was incubated with SSM and CD specfrapolarimetric studies were performed. FIG. 6 (CD spectra analysis of VP (dotted line, grey), and VIP (dotted line, black) in saline compared to VP (solid line, grey), and VIP (solid line, black) in the presence of micelles. Spectrums are an average of 9 accumulations/sample) shows the CD spectra of vasopressin in saline and in the presence of SSM in comparison with VIP spectrums. The spectra indicates that the VP in saline and in the presence of SSM has a similar spectrum with a minima at 204 nm suggesting a primarily random coil conformation, in both cases.

As anticipated vasopressin had no significant changes in its conformation due to the presence of phospholipid micelles, most likely due to its higher affinity to aqueous medium than lipid environment and/or its inflexibility which prevents insertion or penetration into the micellar core.

Therefore, the conformational studies indicate that peptide molecules must be flexible to change its conformation and have an affinity to hydrophobic environment in order to penetrate into the micellar core or lipid bilayer. Furthermore, the negative charge on the PEG-DSPE most likely facilitates the peptide—phospholipid interaction by providing electrostatic attraction. Thus, the CD spectra studies indicate that the VIP most likely enters the hydrophobic micellar core or liposomal bilayer initially due to electrostatic attraction followed by the stable α-helix conformation, which causes the VIP to be in its active conformation for in vivo activity.

### EXAMPLE 8

According to this example, the vasorelaxant effects of VIP in a SSM were determined according to the following method. Specifically, adult male golden Syrian hamsters were purchased from Sasco (Omaha, Nebr.). Adult male hamsters with spontaneous hypertension and their normotensive controls were purchased from the Canadian Hybrid Farms (Halls Harbour, NS, Canada). Hypertensive animals have been identified after cross-breeding of hamsters with hereditary cardiomyopathy and normal golden Syrian hamsters. These albino animals have previously been used in our laboratory (Rubinstein et al., Biochem. Biophys. Res. Commun. 183:1117-1123 (1992); Artwohl et al., FASEB J. 10:A629 (1996)). Animals were anesthetized with pentobarbital sodium (6 mg/100 g body weight, i.p.). A tracheotomy was performed to facilitate spontaneous breathing. A femoral vein was cannulated to inject supplemental anesthesia during the experiment (2-4 mg/100 g body weight/h). Body temperature was maintained constant (37-38° C.) and monitored via heating pad and a feed back controller throughout the experiment.

The bioactivity of the VIP in SSM by diffusing it in situ was determined by visualization of the microcirculation of the hamster cheek pouch. The microcirculation of the cheek pouch was visualized locally by a method previously developed in our laboratory (Suzuki et al., Life Sci. 57:1451-1457 (1995); Suzuki et al., Am. J. Physiol. 271:R393-R397 (1996); and Suzuki et al., Am. J. Physiol. 271:H282-H287 (1996)). Briefly, the left cheek pouch was spread over a small plastic baseplate, and an incision was made in the outer skin to expose the cheek pouch membrane. The connective avascular tissue layer was removed, and a plastic chamber was placed over the baseplate and secured in place by suturing the skin around the upper chamber. This forms a triple-layered complex: the baseplate, the exposed cheek pouch membrane, and the upper chamber. After these initial procedures, the hamster is transferred to a heated microscope stage. The chamber was connected to a reservoir containing warmed bicarbonate buffer (37-38° C.) that allowed continuous suffusion of the cheek pouch. The buffer was bubbled continuously with 95% N₂-5% CO₂ (pH 7.4). The chamber was also connected via a three way valve to an infusion pump (Sage Instruments, Cambridge, Mass.) that allowed constant administration of drugs into the suffusion buffer.

The cheek pouch microcirculation was epi-illuminated with a 100-W mercury light source and viewed through a microscope (Nikon, Tokyo, Japan) at a magnification of ×40. The image was projected through the microscope and into a closed-circuit television system that consisted of a low-light TV camera, monitor and videotape recorder (Panasonic, Yokohama, Japan). The inner-wall diameter of second order arterioles (44-62 mm), which modulated vascular resistance in the cheek pouch, (Raud, Acta Physiol. Scand. (Suppl.) 578:1-58 (1989); Suzuki et al., Life Sci. 57:1451-1457 (1995); Suzuki et al., Am. J. Physiol. 271:R393-R397 (1996)) was measured from the video display of the microscope image using a videomicrometer (VIA 100; Boeckeler Instruments, Tucson, Ariz.). Magnification calibration of the video system was carried out with a microscope stage micrometer to give microvascular dimensions in micrometers. Clarity on the video monitor screen and location within the arteriolar branching pattern in the cheek pouch were the parameters used to determine the vessels chosen for observation. In some experiments, animals were used in more than one treatment group once measures of arteriolar diameter from previous interventions returned to baseline (see experimental protocols).

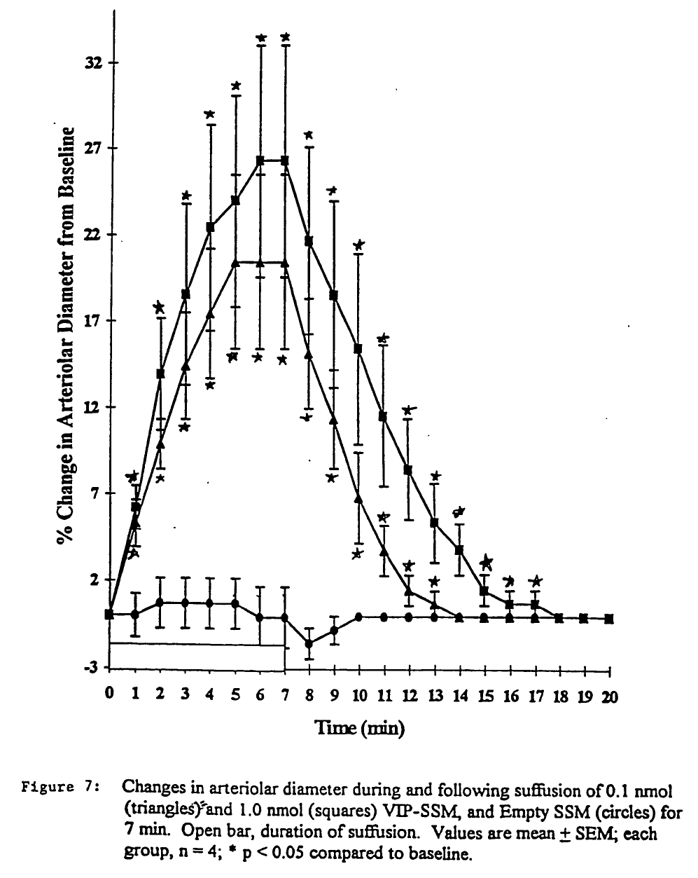

Suffusion of 0.1 nmol and 1.0 nmol of VIP in sterically stabilized phospholipid micelles (SSM) for 7 min induced a significant, concentration dependent, and prolonged vasodilation on the arterioles of the hamster cheek pouch microcirculation. There was an increase in arteriolar diameter of 20.2±2.4%, and 24.5±1%, respectively, from baseline values (FIG. 7; mean±SEM; each group, n=3; p<0.05). Significant vasodilation was observed within 2 min from the start of suffusion and was maximal within 4 min. Arteriolar diameter returned to baseline 7 min (0.1 nmol) and 11 min (1.0 nmol) after VIP-SSM suffusion was stopped. Empty SSM and native VIP alone showed no significant effects on the arteriolar diameter (FIG. 7: Changes in arteriolar diameter during and following suffusion of 0.1 nmol (triangles) and 1.0 nmol (squares) VIP-SSM, and Empty SSM (circles) for 7 min. Open bar, duration of suffusion. Values are mean±SEM; each group, n=4;* p>0.05 compared to baseline.).

The results of the vasorelaxant study showed that the suffusion of VIP in SSM onto the in situ hamster cheek pouch was associated with significant, concentration-dependent and prolonged vasodilation. This prolonged activity of VIP in SSM is surprising since micelles are dynamic and would disintegrate upon suffusion. Thus, the prolonged activity indicates stabilization of the micelles possibly by the presence of VIP that may lead to a formation of VIP-phospholipid complex by hydrophobic interactions, that keeps the micelles intact for a longer period of time. The long lasting activity of VIP-SSM can be attributed to the successful prolonged circulation of the carrier, combined with a stable loading of the peptide, leading to the controlled release of the product. Furthermore, the smaller size of the SSM compared to SSL may additionally increase the circulation time and provide a longer duration of action. In addition, because of the small size of SSM, they are able to migrate into regions inaccessible to liposomes, thereby increasing their biodistribution.

In experiments carried out with the vasoconstrictor peptides angiotensin II and galanin, the same type of increase in biological activity was detected when the peptides were associated with “S1” micelles prepared as described below in Example 11. With angiotensin II at a dose of 0.06 nmol in micelle formulations, vasoconstriction ranged from two to four fold greater than angiotensin II in saline buffer alone. With galanin at a dose of 0.1 nmol in an S1 (Example 11) micelle preparation, vasoconstriction was approximately two times greater than galanin in buffer. At a higher dose (1.0 nmol), the effect of galanin in micelles was still significantly greater (approximately 33%) than galanin in buffer.

### EXAMPLE 9

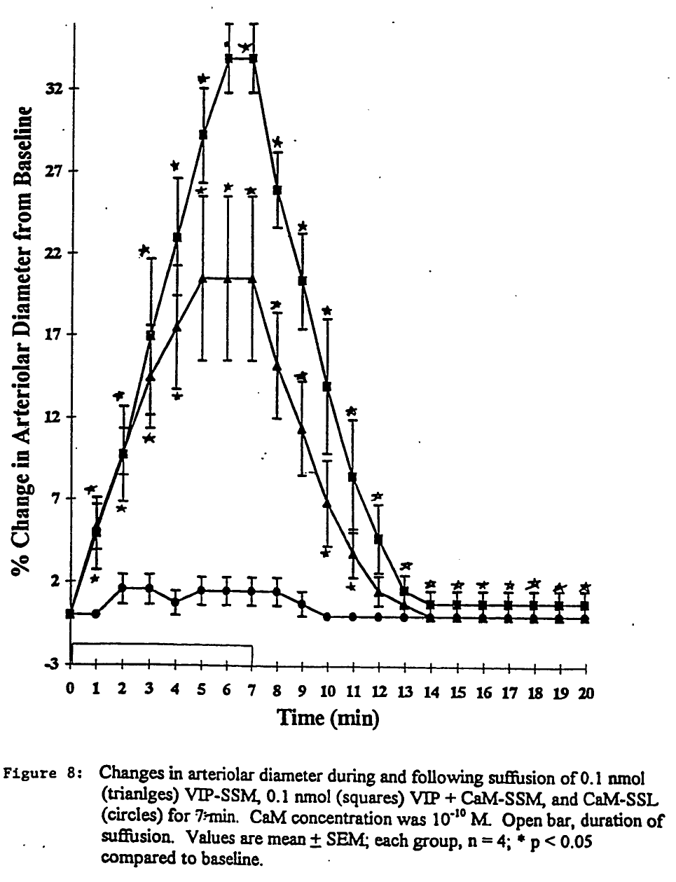

According to this example, the role of calmodulin (CaM) on the vasorelaxant effects of VIP in a SSM were determined according to the methods of example 7. Specifically, a suffusion of 0.1 nmol of VIP+CaM in SSM for 7 min elicited a significant, and prolonged potentiation of VIP-SSM induced vasodilation on the arterioles of the hamster cheek pouch microcirculation. There was an increase in arteriolar diameter of 40±1% from baseline values (FIG. 8; mean±SEM; each group, n=4; p<0.05). Significant vasodilation was observed within 2 min from the start of suffusion and was maximal within 5 min. Arteriolar diameter returned to baseline 8 min after VIP±CaM-SSM suffusion was stopped. Empty CaM-SSM and native VIP alone showed no significant effects on the arteriolar diameter (FIG. 8: Changes in arteriolar diameter during and following suffusion of 0.1 nmol (triangles) VIP-SSM, 0.1 nmol (squares) VIP+CaM-SSM, and CaM-SSL (circles) for 7 min. CaM concentration was 10⁻¹⁰ M. Open bar, duration of suffusion. Values are mean±SEM; each group, n=4;* p<0.05 compared to baseline.)

The results of these studies showed that suffusion of VIP+CaM in SSM potentiated the significant, concentration-dependent, and prolonged vasodilation of the cheek pouch circulation induced by VIP in SSM. These potentiating effects may partly be due to the calmodulin interactions with phospholipid and thus exposing their hydrophobic-protein binding region. Furthermore, this hydrophobic region promotes the α-helix conformation of the VIP due to an increase in the hydrophobic environment of the SSM. The amplification of VIP in the α-helical structure would provide enhanced induction of the receptor-reactive complex and may also increase 2^{nd} messenger actions of VIP by promoting direct contact with membranes and membrane-bound proteins. Moreover, the addition of CaM may decrease the CMC causing the increase in micelle number, which further increases the hydrophobicity of the solution and leads to an amplification of α-helix structure. This increase in the amount of active VIP available may be the mechanism by which CaM potentiates the vasodilation of VIP in SSM.

### EXAMPLE 10

According to this example, the hypotensive effects of the SSMs of the previous examples on mean arterial pressure are determined.

In order to determine mean arterial pressure a catheter is inserted into the left femoral artery of the hamster to record systemic arterial pressure and heart rate using a pressure transducer and a strip-chart recorder (Model 260, Gould Instrument Systems Inc., Valley View, Ohio). Continuous anesthesia of the animals limited the monitoring of mean arterial pressure to 6 hours. The cannulated femoral vein was used to administer the products injected intravenously. VIP in SSM (0.1 nmol) is injected intravenous (i.v.) in hypertensive hamsters for 1 min at a rate of 0.5 ml/min. VIP only (0.1 nmol) and empty SSM (concentration equivalent to 0.1 nmol if VIP had been encapsulated, i.e., ˜18 nmol phospholipids) are also injected in hypertensive hamsters. The mean arterial pressure (MAP) was calculated every five min for 6 h, and variations associated with the injection of anesthesia were not considered.

According to one aspect of the example the effects of VIP-SSM when administered intravenously in normotensive hamsters is studied. VIP-SSL (0.1 nmol), empty SSL, and VIP only (0.1 nmol) are injected in normotensive hamsters at the same rate as in hypertensive hamsters. The temperature of the hamster is maintained by using a hot water pad placed under the hamster. Intravenous administration of the VIP-SSM in hamsters with spontaneous hypertension is expected to elicit significant and prolonged hypotensive effects.

### EXAMPLE 11

According to the present example, an alternative method for producing micelles comprising amphiphilic compounds was designed. This alternative method of preparation, in comparison to the method described in Example 1, is more readily amenable to safe and large scale production of micelles of the invention. The method is exemplified as follows using human galanin, a 30 amino acid neuropeptide with mostly inhibitory, hyperpolarizing biological activity.

DSPE-PEG (16.5 mg, molecular weight 2748.01) was placed in a 20 ml glass vial and 6 ml saline buffer was added to give a final DSPE-PEG concentration of 1.0 μmol/ml. The mixture was vortexed for one minute until the solution was clear, after which the vial was topped with argon and sealed with parafilm. The mixture was allowed to stand at room temperature for one hour or until the bubbles rose out of the mixture. The resulting micelle solution was designated “S1”. Twelve μg human galanin (molecular weight 3158.1) was placed in a polypropylene tube and 5 ml of the S1 micelle preparation was added to the tube giving a final galanin concentration of 1 nmol/1.4 ml. The mixture was vortexed for ten seconds and incubated at room temperature for two hours. The size of the resulting galanin containing micelles, 17 to 20 nm, was measured by QELS as described in Example 3.

### EXAMPLE 12

According to the present example, micelle composed of two different compositions were prepared and characterized in order to determine an optimal system for increasing solubility of normally water-insoluble compounds. In the first system, micelles were composed of DSPE-PEG and PC. When DSPE-PEG is mixed with phosphatidylcholine (PC) in aqueous medium, mixed micelles are formed instead of liposome bilayers. In the second system, micelles were formed using PC in combination with a representative bile salt, sodium taurocholate (Sigma). When small molecular weight surfactants, such as bile salts, are mixed with DSPE-PEG, formation of spherical mixed micelles can also be detected. The purpose of this study was (i) to compare the effect of DSPE-PEG and bile salts on phosphatidylcholine (PC) capacity to form mixed micelles; (ii) to examine and compare characteristics of the resulting mixed micelles, including micelle-to-vesicle-transition upon dilution; and (iii) to compare solubilization potential of the two micelle systems.

For both compositions, aqueous detergent-phospholipid mixed micelle stock solutions were prepared by co-precipitation [Alkan-Önyüksel, et al., Pharm. Res. 11:206-212 (1994)]. Briefly, egg L-alpha-phosphatidylcholine type XIII-E (Sigma) was combined with either DSPE-PEG 2000 (Avanti) or sodium taurocholate (Sigma) at a PC/detergent molar ration of 0.7 and 0.8, respectively. The mean hydrodynamic diameter of the micelles was measured by quasi-electric light scattering (see Example 3) and small angle neutron scattering (SANS) [Hjelm, et al., J. Phys. Chem. 96:8653-8661 (1992)]. In assessing micelle-to-vesicle-transition, the two stock solutions were diluted rapidly in one step and micelle-to-vesicle-transition curves were determined by following the change in aggregate size upon aqueous dilution at room temperature in the presence or absence of counter ions.

The mean size of the DSPE-PC micelles was consistently larger (17 to 22 nm) than micelles containing bile salts (3 to 5 nm). Aqueous dilutions of bile salt mixed micelles resulted in a detectable micelle-to-vesicle-transition, however, no transition was observed under similar conditions with the DSPE-PEG/PC mixed micelles. Bile salts, when added to preformed PC liposome dispersions, resulted in formation of mixed micelles from the pre-existing liposomes, whereas addition of DSPE-PEG to PC liposomes did not demonstrate any vesicle to micelle transition. These results suggest that the hydrophilic PEG component of the DSPE-PEG molecules prevent micelle/micelle or bilayer/micelle interactions. Since DSPE-PEG did not solubilize liposomes, it is anticipated that it will not solubilize a plasma membrane. This result indicated the potential for lower plasma membrane toxicity of DSPE-PEG than bile salts.

Solubilization potential of both micelle compositions for a model drug was measured by HPLC after separation of the unincorporated drug. For purposes of this series of experiments, progesterone, virtually insoluble in an aqueous environment, was chosen as the model drug.

In addition, solubility of progesterone in DSPE-PEG micelles was approximately five to ten times larger than bile salt micelles (from 21 μg/ml to 198±7 μg/ml) for the same total lipid concentration, thereby suggesting that DSPE-PEG micelles have a greater potential as an efficient vehicle for insoluble drugs. However, this dispersion contained both SSM (at approximately 17 nm) and SSC (at approximately 150 nm).

### EXAMPLE 13

According to the present example, enhanced solubility of normally water-insoluble compounds was further investigated using a DSPE-PEG micelle composition. In addition, a method for preparing micelles comprising a targeting agent in addition to an encapsulated water-insoluble compound was designed. Drug solubility was determined as follows.

Active drug loading was carried out by adding an excess of drug in powder form to a polyethylene microfuge tube containing PC/bile salt or DSPE-PEG prepared using the film method as previously described in Example 1. Excess drug was removed by centrifugation and the supernatant was analyzed by HPLC. In the case of progesterone, HPLC conditions included a YMC-CN (A-503, 250×4.6 mm inner diameter) column, a mobile phase comprising acetonitrile and water (40:60), and a flow rate of 1.5 ml/minute. HPLC eluent was measured with adsorption at 254 nm. For PC/bile salt mixed micelles, the progesterone to lipid ratio was determined to be 0.0156. For DSPE-PEG micelles, the progesterone to lipid ratio was found to be 0.17.

Results indicated that progesterone (essentially insoluble in water as discussed above) in 10 mg/ml DSPE-PEG was soluble up to 198.5 μg/ml. This result was consistent with results using betullinic acid, sparingly soluble in water (see Merck Index, 12th Edition, p. 1213), which was soluble up to 200 μg/ml in 10 mg/ml DSPE-PEG. In similar experiments with betulinic acid (also insoluble as defined in USP), solubility was calculated at 250 μg/ml in either SSM or SSC.

In design of a targeted drug delivery system comprising micelles, a desired compound is incorporated in micelle compositions as described above. The resulting micelle compositions are then incubated with an amphiphilic compound to allow incorporation of the compound at, and into, the micelle surface as described in Example 1. The membrane associated compound in this arrangement acts as a targeting agent for the entire micelle composition to be delivered to, for example, a receptor for the membrane associated compound. In an alternative approach, the amphilic compound is linked, preferably through covalent modification, to one or the lipid components of the micelle. Through either of these mechanisms, the micelles can carry and deliver the incorporated drug to a target cell or tissue type expressing the cognate receptor.

For example, breast cancer cells express higher levels of VIP receptor than normal breast cells. Micelles comprising membrane associate VIP will therefore preferentially bind to a breast cancer cell rather than a normal cell. Since paclitaxel (Taxol®) has been shown to kill breast cancer cells, incorporating paclitaxel (Taxol®) in a VIP/micelle provides a targeted drug delivery for selective killing of the carcinoma cell type.

### EXAMPLE 14

According to this aspect of the invention, the effect of infusion of enthelin-1 (ET-1) alone or in a SSM formulation on mean arterial pressure (MAP), cardiac output(CO), total peripheral resistance (TPR), and regional blood circulation in anesthetized rats was examined using a radioactive microsphere technique. SSM with or without ET-1 were prepared according to the method described in Example 11 using DSPE-PEG in saline. Treatments for individual groups consisted of: (i) control, SSM at 2.7 mg/ml (n=6); (ii) ET-1 infusion at 50 ng/kg/min (n=5), and (iii) ET-1 at 50 ng/kg/min in SSM (n=8). Drugs were infused at 0.1 ml/min for 30 minutes.

Results showed that SSM did not affect MAP, CO, TPR or gastrointestinal tract (GIT) blood flow, however, an increase in blood flow to the kidneys (approximately 25%) and brain (approximately 19%) was observed compared to baseline. Blood flow decreased and vascular resistance increased in the kidney, GIT, and brain. ET-1 in SSM produced a significantly marked cardiovascular effect as compared to ET-1 alone. The increase in TPR was 102% in the ET-1 group and 227% in the group treated with ET-1 in SSM. Renal vascular resistance was 76% in the ET-1 group and 281% in the group treated with ET-1 in SSM. However, in brain, vascular resistance was 62% in the ET-1 group and 20% in the group treated with ET-1 in SSM. These results indicated that changes in MAP, TPR and regional vascular resistance are potentiated by ET-1 in SSM.

### EXAMPLE 15

According to this aspect of the invention, the ability of micelle products of the invention to enhance cellular viability following cryopreservation was examined. In this experiment, cells were incubated with either DMSO, DSPE-PEG micelle products, or DSPE-PEG micelles products including VIP for 30 minutes prior to storage for 48 hours in liquid nitrogen. Follow removal from the liquid nitrogen, cells were thawed and viability was measured using Trypan blue using standard techniques.

Results indicated that cell viability following treatment with micelles, with or without associated VIP, was equal to or greater than cell viability following treatment with DMSO. Because DMSO is well known and routinely used in the art for cell cryopreservation, these results indicate that micelles can afford equal or better protection, thereby providing an alternative protective agent for cell storage.

### EXAMPLE 16

In this example the bioactivity, receptor specificity and conformation of secretin self-associated in sterically stabilized phospholipid micelles (SSM) were studied.

DSPE-PEG2000 was dissolved in saline. Secretin as a dry powder, was added and incubated at room temperature for 2 hours. The cheek pouch of anesthetized adult male Syrian hamsters were prepared for intravital microscopic study of the microcirculation as described in Sejourne et al., Am. J. Physiol., 273:R287-R292 (1997). Mean arterial pressure and heart rate were monitored and did not change significantly throughout the duration of the experiments. The diameter of second-order arterioles in the cheek pouch was determined during suffusion of buffer for 30 minutes followed by suffusion of secretin (5 nmol) alone or in SSM onto the cheek pouch for 7 minutes. In addition, empty SSM were suffused for 7 minutes in a similar fashion. At least 2 hours elapsed between subsequent suffusions. Arteriolar diameter was measured immediately before and every minute during and after suffusion of drugs for one hour. Secretin in SSM (5 nmol) was suffused for 7 minutes. Two hours thereafter, VIP₁₀₋₂₈ (50 nmol), a selective VIP receptor antagonist was suffused for 30 minutes before and during repeated suffusions of secretin (5 nmol) alone or in SSM for 7 minutes. Arteriolar diameter was determined as described above. The conformation of secretin in saline and SSM (5 μM) was determined by circular dichroism (CD) using a JASCO J-710 spectropolarimeter at room temperature.

Suffusion of secretin in saline and in SSM (5 nmol) was associated with a significant and prolonged increase in arteriolar diameter, (10.3±0.6%, 14.4±0.7% [mean±SEM] increase from baseline, respectively; n=4; p,0.05). The effects of secretin in SSM on arteriolar diameter over time was significantly greater than that evoked by secretin alone (n=4; p,0.05). Suffusion of empty SSM had no significant effects on arteriolar diameter. Suffusion of VIP₁₀₋₂₈ alone had no significant effects on arteriolar diameter. However, it abrogated secretin- and secretin in SSM-induced vasodilation (n=4; p<0.05). CD spectroscopy revealed that secretin is unordered in saline. However, it assumes appreciable α-helix conformation in the presence of SSM.

The results indicated that association of secretin with SSM amplifies its vasoactive effects in vivo in the intact peripheral microcirculation. This response is most likely modulated by stimulation of VPAC₁ receptors and conformation transition of secretin molecule in SSM to α-helix. The results indicate that secretin in SSM could be the preferred formulation for human use.

### EXAMPLE 17

According to this example, the ability of DSPE-PEG 5000 to interact with and stabilize IL-2 in aqueous medium was assessed. Protein stability was determined by circular dichroism and fluorescence spectroscopy for secondary and tertiary structure determinations, respectively, turbidity by UV, and visual appearance testing.

IL-2 is a well characterized hydrophobic protein containing a single tryptophan within a four α-helical bundle. These properties render IL-2 ideal for interacting with phospholipids and characterization by fluorescence spectroscopy in that the tertiary structure may be monitored by a shift in the emission wavelength. The isoelectric point (pI) of IL-2 is 7.05. At this pH the protein is chemically most stable but physically least stable. In this example, IL-2 was stored in the presence of DSPE-PEG 5000 at the pI of the cytokine so that the protein will be unfolded and electrically neutral to provide a physically interactive environment.

In order to determine the ability of DSPE-PEG 5000 to interact with and stabilize IL-2 in aqueous medium samples were prepared. To obtain the protein in the native state, pure lyophilized recombinant human IL-2 (no excipients) was dissolved in 15 mM sodium acetate at pH 5.0. DSPE-PEG 5000 micelles (100 μM) were prepared by adding 100 mM Tris buffer at pH 7.1, to dry DSPE-PEG 5000. The phospholipid mixture was vortexed for 2 minutes and then sonicated under vacuum for 5 minutes. Micellar size (˜25 nm) was assessed in a Nicomp 380 Particle Size Analyzer prior to the addition of protein. Protein was added to the micellar solution or to Tris buffer alone. The final concentration of IL-2 in all protein samples was 0.12 mg/ml. DSPE-PEG 5000 was 70 μM in all DSPE-PEG 5000 samples. Final pH of the solution was between 7.0 and 7.1. DSPE-PEG 5000 in buffer and buffer alone were included as controls. Samples were stored in type I, glass vials with FluoroTec®coated stoppers and stored at 5° C. and 25° C. for 28 days. Experiments were carried out in duplicate.

Sample analysis was conducted by circular dichroism (CD) for changes in secondary structure, fluorescence spectroscopy (excitation 295 nm, emission 305-500nm) for changes in tertiary structure, UV (A360) for turbidity, and visual appearance (color, clarity and precipitate). CD spectra were analyzed by SELCON (Softsec version 1.2, 1996) to determine % α-helical content.

Visual turbidity was noted upon initial reconstitution of the lyophilized protein. However, the turbidity observed in the protein solution decreased upon addition into DSPE-PEG 5000 as compared to similar dilution with buffer alone. 100 μM DSPE-PEG 5000 micelles in 100 mM Tris buffer (pH 7.1) yielded a clear, colorless solution. The turbidity observed in the IL-2/DSPE-PEG 5000 samples at 25° C. increased at the same rate as that observed in the DSPE-PEG 5000/buffer samples, suggesting that the increased turbidity was caused primarily by degradation of DSPE-PEG 5000. IL-2/DSPE-PEG 5000 samples stored at 5° C. remained unchanged over the 28-day period studied.

Secondary structure of IL-2 was preserved in the presence of DSPE-PEG 5000 for the entire study whereas IL-2 in buffer alone retained <50% of the original α-helical structure after 7 days in solution regardless of storage temperature. No peak shift in fluorescence was observed between IL-2/DSPE-PEG samples and IL-2/buffer samples. However, fluorescence intensity of IL-2/DSPE-PEG 5000 samples was significantly greater than IL-2/buffer samples. The fluorescence from DSPE-PEG 5000 in buffer alone does not explain this difference. The difference in fluorescence intensity is likely due to the greater amount of aggregate and precipitate present in IL-2/buffer samples. A significant amount of precipitate was noted by visual appearance in the IL-2/buffer samples after 3 days storage.

Results indicated that IL-2 interacts with DSPE-PEG 5000 (molar ratio W˜9:1) at the pI of the protein. This interaction at pH 7 increases the physical stability of IL-2. These results suggested that relatively safe, pegylated phospholipids can be used to stabilize IL-2 in aqueous medium for at least 28 days at 5° C. The underlying mechanism of interaction remains unclear.

### EXAMPLE 18

According to this example, micelle compositions of the invention were further characterized. Particularly, the physiochemical properties of sterically stabilized micelles prepared with DSPE conjugated to molecular weight 2000, 3000, and 5000 PEG were analyzed. The critical micelle concentration (CMC) of phospholipids was determined at pH 7.4 and 25° C. using a water-insoluble fluorescent probe (1,6-diphenyl-1,3,5-hexatriene). Micellar size was determined by quasi-elastic light scattering. Solubilization potential of micelles was determined using diazepam as a model hydrophobic drug and RP-HPLC.

As a result, CMC of DSPE-PEG micelles increased from 0.5 to 1.5 μM range as molecular weight of PEG increased from 2000 to 5000. Mean hydrodynamic diameters (±SEM) of micelles were 16.8±0.3, 20.3±0.6 and 23.9±2.1 nm for DSPE-PEG 2000, 3000, and 5000, respectively. Furthermore, maximal concentration (±SD) of diazepam solubilized in DSPE-PEG 200, 3000, and 5000 was 288.97±7.51, 224.26±6.22 and 195.92±19.73 μg/ml at a constant concentration of phospholipid (1 mM), respectively.

These results indicated that shorter PEG chain length of DSPE-PEG results in smaller micellar size and lower CMC with increased solubilization potential for insoluble drugs. This suggests that DSPE-PEG 2000 micelles are better solubilizers for small hydrophobic molecules, which could be related to an increase in the number of micelles/molar lipid concentration.

### EXAMPLE 19

According to this example, DSPE conjugated with 1, 2, 3 or 5 KDa PEG in solution, alone or mixed with egg yolk phosphatidylcholine (EYPC) were studied by static (SLS) and dynamic light scattering (DLS).

SLS and DLS was used to study micelles in DSPE conjugated with PEG of nominal molecular weight 1, 2, 3 or 5 KDa, either alone or with 25 mole % EYPC, as a function of total phospholipid concentration. The phospholipids were dissolved in methanol and dried as a film. The films were dissolved in 10 mM HEPES buffer, pH 7.4, 0.15 NaCl with agitation. The samples were then flushed with nitrogen, sealed and incubated in the dark at room temperature for 48 hours. Samples were passed through a 0.2 μ filter to eliminate dust.

The apparatus was configured to measure SLS and DLS as a function of momentum transfer, Q. Q is related to the scattering angle, 2θ, wavelength, λ=632.8, and medium index of refraction, n, as,

$$Q = \frac{4 Π n}{λ} ( sin θ )$$

Correlation functions are measured using ALV-5000 Multiple Tau Digital Correlator over lag times between 2×10⁻⁷ and 10 s. Multiple angle scattering intensity and correlation functions over a large dynamic range allow detailed characterization of micelle size, shape and polydispersity.

The Guinier approximations for SLS of globular particles,

$$I ( Q ) = Δ M exp - • \underline{{Q}^{2} {R}_{g}^{2}} • , \\ •3•$$

and equivalent forms for rods and sheets (Hjelm et al., J. Phys. Chem., B104:197 (2000), are used to make estimates of the particle radius of gyration, R_{g} in the domain R_{g}Q<1.3 R_{g} and shape. DLS gives estimates of the diffusion coefficient, D, of particles in a media of viscosity η, by measurements of the time-dependent correlation function. D can be used to estimate the particle hydrodynamic radius, R_{H} through the Stokes-Einstein equation,

$${R}_{H} = \frac{k T}{6 Π η D}$$

These results indicated that DSPE-PEG 1000 does not form micelles in either simple or mixed surfactant solutions. DSPE-PEG at 2, 3, and 5 KDa formed micelles at 1.1 mM and lower with and without EYPC. With EYPC the micelles were considerably larger. At higher concentrations DSPE-PEG/EYPC mixtures form an anistropic phase. The characterization of particular forms met the expectations that when EYPC is incorporated into the simple DSPE-PEG micelles, the particular curvature and shape will change to give a bigger hydrophobic core and therefore the solubilization potential of phospholipid micelles will improve. The results indicate that the size can be controlled by the addition of a second phospholipid. This shows that the approach may be useful in developing micellar drug delivery systems.

### EXAMPLE 20

According to this example the therapeutic uses of the invention are analyzed. Previously, sterically stabilized liposomes (SSL) were prepared with VIP non-covalently associated on their surface. However, these liposomes were not able to actively target to breast cancer in rats in situ. In this example, the need to conjugate VIP covalently to SSL is studied and the targeting ability of VIP-SSL to n-methyl nitrosourea (MNU)-induced rat breast cancer in vitro is tested.

DSPE-PEG₃₄₀₀-NHS [1,2-dioleoyl-sn-glycero-3-phosphoethanolamine-n-[poly(ethylene glycol)]-N-hydroxy succinamide, PEG M_{w} 3400] and polyethylene glycol (M_{w} 2000) conjugated distearyl phosphatidylethanolamine (DSPE-PEG₂₀₀₀) were obtained from Shearwater Polymers, Inc. (Huntsville, Ala.). BODIPY-Chol (flourescent cholesterol) was obtained from Molecular Probes Inc. (Portland, Oreg.). Fluo-VIP™ (Portland, Oreg.). Fluo-VIP™ fluorescein labeled VIP) was purchased from Advanced Bioconcept (Montreal, Quebec, Canada). VIP (human/rat) was synthesized, using solid-phase synthesis by Protein Research Laboratory at Research Resources Center, University of Illinois at Chicago. Egg-phosphatidylcholine (PC) and cholesterol (CH) were obtained from Sygena (Switzerland). Virgin female Sprague-Dawley rats (˜140 g body weight) were obtained from Harlan (Indianapolis, Ind.).

In conducting research using animals, the investigators adhered to the Institutional Animal Care Committee guidelines and to the Guide for the Care and Use of Laboratory Animals of the Institute of Laboratory Animal Resources, National Research Council.

An activated DSPE-PEG (DSPE-PEG₃₄₀₀-NHS) was used to conjugate VIP to DSPE-PEG₃₄₀₀. This reaction takes place between amines and NHS group, which acts as the linking agent. VIP and DSPE-PEG₃₄₀₀-NHS in the molar ratio of 1:5 (VIP:DSPE-PEG₃₄₀₀-NHS) were dissolved separately in 0.01 M isotonic HEPES buffer, pH 6.6. DPSE-PEG₃₄₀₀-NHS solution was added in small increments over 1-2 min to the VIP solution at 4° C. and then stopped by adding glycine solution to the reaction mixture to consume the remaining NHS moieties. The conjugation was tested using SDS-PAGE and subsequent staining with first Coomassie Blue R-250 and then silver stain. The VIP conjugated to DSPE-PEG₃₄₀₀ (DSPE-PEG₃₄₀₀-VIP) was subsequently used to prepare fluorescent VIP-SSL.

Breast cancer was induced in rats with MNU as previously described in S. Dagar et al., Breast Cancer Treatment, in press and G. O. Udeani et al., Cancer Research, 57:3424-3428 (1997). Briefly, virgin female Sprague-Dawley rats, 36 days old, weighing ˜140 g, were anesthetized with ketamine/xylazine (13.3/1.3 mg per 100 g body weight, i.m.). Each animal received a single intravenous injection of MNU (50 mg/kg body weight) in acidified saline (pH 5.0), via the tail vein. The rats were weighed weekly. They were palpated every week, starting at 3 weeks post-MNU administration. Palpable mammary tumors were detected within 100-150 days after injection.

For testing the in vitro binding, BODIPY-Chol (a non-exchangeable fluorescent probe) containing liposomes, were prepared with film rehydration-extrusion method, as described in S. Dagar et al., Pharm. Sci., 1:S-294 (1998) and M. Patel et al., Proc. Int. Symp. Control. Rel. Bioact. Mat., 24:913-914 (1997) but incorporated the probe at 1:1500 molar ratio (lipid:probe) in the lipid mixture. Egg phosphatidylcholine (PC), cholesterol (CH), DSPE-PEG₂₀₀₀ and dipalmitoyl phosphatidylglycerol (DPPG) in the molar ratios of PC:DPPG:DSPE-PEG₂₀₀₀:CH of 0.50:0.10:0.03:0.35 were used to form the sterically stabilized liposomes by film rehydration and reconstitution using isotonic, 0.01 M HEPES buffer (pH 6.6). This was followed by extrusion through polycarbonate filters (100 nm) using a Liposofast® (Avestin Inc., Canada) extruder. The size of final liposomes was ˜140 nm as determined using quasi-elastic light scattering (NICOMP 370, Particle Sizing Systems, Santa Barbara, Calif.). DSPE-PEG₃₄₀₀-VIP was inserted into these fluorescent liposomes by overnight incubation at 4° C. to form fluorescent VIP conjugated sterically stabilized liposomes (VIP-SSL).

The rats were euthanized by exposure to carbon dioxide in a closed chamber. Normal and cancerous breast tissue were excised, frozen immediately in liquid nitrogen and stored at −70° C. until use. The frozen breast tissue was cut into 20-mm sections and mounted on microscopic slides. They were then fixed with 4% formaldehyde and allowed to air-dry for 10 min. Adjacent 5 mm thick frozen tissue sections, were stained with hemotoxylin and eosin to confirm the presence or absence of cancer in the breast tissue. The presence of VIP-R in these rat breast cancer tissues was confirmed using a fluorescent VIP, FluoVIP™ as described in S. Dagar et al., Breast Cancer Res. Treatment (2000) in press. Twenty micormeter sections of MNU-induced rat breast cancer tissues were cut using a cryotome, placed on a slide, fixed with 4% formalin for 20 min., and then air-dried for 1Omin. The BODIPY-Chol containing VIP-SSL were added to the sections and incubated for 1 h at room temperature. At the end of the incubation period, the slides were washed with 0.01 M isotonic HEPES buffer, pH 6.6, four times for 60 s each. The slides were then observed with a Zeiss Camera (Carl Zeiss Inc., Thomwood, N.Y.) and photographed. All photographs were taken with a 2 min exposure using Kodak Elite Chrome 400 photographic film. The VIP-SSL were compared to SSL without VIP or with non-covalently associated VIP and the difference in number of fluorescent liposomes present on the tissue indicated the difference in attachment of VIP-SSL to MNU-induced rat breast cancer tissues.

The reaction conditions were optimized after systemic variation of pH, reaction time, reaction temperature, molar ration of VIP: DSPE-PEG₃₄₀₀-NHS and stirring rate. It was found that the conditions of reaction (2 h at 4° C., pH 6.6, gentle stirring and 1:5 molar ratio) currently used gave the best results. Therefore, the subsequent experiments were done using these optimized conditions. The stained gel (SDS-PAGE) of the conjugation mixture showed that most of the product is 1:1 conjugate of VIP and DSPE-PEG₃₄₀₀ (DSPE-PEG₃₄₀₀—VIP), and free VIP and 1:2 conjugate of VIP and DSPE-PEG₃₄₀₀ exist at much lesser extent as compared to 1:1 DSPE-PEG₃₄₀₀-VIP conjugate. Furthermore, the fluorescence microphotographs of breast cancer tissues indicated that more VIP-SSL were attached to MNU-induced rat breast cancer tissue sections while SSL without VIP or with non-covalently associated VIP, showed no significant attachment.

In this experiment VIP was successfully conjugated to DSPE-PEG₃₄₀₀ and incorporated into preformed sterically stabilized liposomes to form a VIP-SSL construct. The results showed the feasibility of this novel construct to actively target to MNU-induced rat breast cancer in vitro.

### EXAMPLE 21

According to this example, VIP-SSM and its therapeutic effects in the treatment of inflammatory disease, such as collagen induced arthritis (CIA) in mice, were further characterized and evaluated. It previously has been shown that the repeated intraperitoneal administration of VIP (5.0 nmol) ameliorates CIA in mice [Delgado et al., Nat Med 7: 563-568, (2001)]. However, its short half-life in vivo has precluded its clinical use. The delivery of VIP in SSM (VIP-SSM) increases its stability, half-life and targets diseased tissues [Onyuksel et al., Pharm Res 16: 155-160, (1999)]. Therefore, this study examined the therapeutic effects of the intravenous delivery of VIP-SSM in mice with CIA.

SSM were prepared as described above in previous examples and by Onyuksel et al. (1999). VIP (1.0 nmol) was incubated with SSM for 2 h at 25° C. Size of VIP-SSM was determined by dynamic light-scattering. The effects of dilution on VIP-SSM were determined by size exclusion chromatography (SEC). Effects of VIP-SSM (1.0 nmol) and VIP in buffer (1.0 nmol and 5.0 nmol) were evaluated in mice with CIA treated on day 22 or 34 post-CIA induction. Clinical arthritis score (CAS) and hind paw thickness (PT) were recorded until day 45 post induction.

Size of VIP-SSM was ˜17nm. SEC showed that VIP-SSM eluted at 8.7 min comparable to thyroglobulin, a protein marker (˜20nm) eluting at 8.6 min. VIP-SSM was the major peak with a minor peak for free VIP at 12.8 min. Upon 10-fold dilution, VIP-SSM eluted as one major peak with no significant increase in free VIP. Preliminary PT and CAS data showed reduced progression of CIA in mice treated on day 22 or 34 with VIP-SSM [days 22 (45.10%) and 34 (58.60%), respectively] compared to VIP in buffer at 1.0 nmol [days 22 (78.09%) and 34 (96.55%), respectively] or VIP in buffer at 5.0 nmol [days 22 (56.37%) and 34 (69.71%), respectively].

This study demonstrated that most of the VIP remained associated with SSM upon dilution. Also, greater efficacy in reducing was shown for VIP-SSM than for VIP in buffer alone at 5 times lesser concentration. These data indicated that VIP-SSM was successful in the treatment of CIP in mice, and suggest that VIP-SSM can be used as a novel therapy for the treatment of other inflammatory diseases as well.

### EXAMPLE 22

According to this example, the therapeutic delivery of α-helix VIP in SSM (VIP-SSM) was tested as a therapeutic for the treatment of inflammatory diseases. The short half-life of and hypotension evoked by VIP has previously precluded its clinical use in the treatment of inflammatory diseases such as rheumatoid arthritis (RA). However, it has been shown that the delivery of VIP-SSM increases the stability, half-life and bioactivity of VIP. Therefore, the purpose of this study was to determine the effects of the intravenous administration of α-helix VIP (VIP-SSM) in mice with collagen-induced arthritis (CIA).

VIP, VIP-SSM (each, 0.5, 1.0 & 5.0 nmol), empty micelles, and buffer were injected into the tail vein on day 22 or day 34 post-CIA induction. Clinical arthritis score (CAS) and hind paw thickness (PT) were recorded until day 45 post-CIA induction. Systemic arterial pressure (SAP) was recorded by tail cuff in restrained mice. We found that PT was increased by 38.34±4.64, 35.36±2.05%, and 26.47±2.94% in α-helix VIP (0.5, 1.0 and 5.0 nmol dose, respectively)-treated mice versus 85.63±6.20%, 78.66±6.44%, and 46.69±6.92% for aqueous VIP (0.5, 1.0 and 5.0 nmol dose, respectively)-treated animals (n=4; p<0.05). CAS reductions were similar to PT after treatment with α-helix VIP- and aqueous VIP-treated mice. Empty micelles had no significant effects on CIA. Unlike aqueous VIP, α-helix VIP had no ignificant effects on SAP.

Collectively, these data showed that low dose intravenous α-helix VIP significantly attenuated CIA in mice with no significant effects on SAP. These results suggest that α-helix VIP delivered in SSM represents a novel therapy for the treatment of inflammatory diseases such as RA.

### EXAMPLE 23

According to this example, sterically stabilized mixed micelles (SS), SSM which are composed of poly(ethylene glycol-2000)-grafted distearoylphosphatidylethanolamine (PEG(2000)-DSPF,), plus egg-phosphatidylcholine (PC) with SSM, were investigated as a novel carrier for the delivery of water-insoluble drugs. SSMM improve the solubilization potential of SSM by increasing the solubilization potential by increasing the hydrophobic core of each SSM by incorporating PC. SSMM, thus as the second generation of SSM, retained all the advantages of SSM while increasing the solubilization capacity of the micelle for a hydrophobic drug. This study investigated the in vitro use of SSMM as an improved drug delivery system for the delivery of the anti-cancer drug, paclitaxel (also known as Taxol®), and compares it to the delivery of paclitaxel in SSM, which has serious formulation problems (Terwogt et al., Cancer Treat Rev. 23:87-95(1997); Kohler and Goldspiel, Pharmacotherapy 14:3-34 (1994); van Zuylen et al., Cancer Chemother Pharmacol. 47:309-318 (2001).

Paclitaxel was solubilized in SSM (P-SSM) and sterically stabilized mixed micelles (P-SSMM) by coprecipitation and rehydration with isotonic 0.01M HEPES buffer, pH 7.4, as follows. Briefly, for simple micelles, paclitaxel and PEG(2000)-DSPE, in a molar ratio of 0.16 was dissolved in methanol. The solvent was then removed by vacuum rotary evaporation under a stream of argon to form a dry film. This dry film was further dried under vacuum overnight to remove any traces of remaining solvent. The dried film was rehydrated with isotonic 0.01M HEPES buffer, pH 7.4. The solution was then flushed with argon, sealed and equilibrated for 12 h at room temperature. The unsolubilized excess paclitaxel was removed by centrifugation at 13,000 g for 5 min to obtain a clear dispersion. The maximum solubility of paclitaxel in the absence of crystal formation was determined in simple micelles of PEG(2000)-DSPE by keeping the phospholipid concentration fixed at 5 mM and systematically reducing the drug concentration (Drug:phospholipid, molar ratios, 0.076, 0.078, 0.082, 0.088) until a single homogenous system was determined as confirmed by a single peak by size analysis.

To prepare SSMM solubilizing paclitaxel, initially various molar ratios of PEG(2000)-DSPE and EPC (90:10, 85:15, 80:20 and 75:25) were coprecipitated along with 500 μg paclitaxel, and the same procedure as described above was followed. The total phospholipid concentration was kept constant at 5 mM. Each formulation was prepared in triplicate. The prepared dispersions were then characterized for their size and morphology and assayed for their drug content. The optimal formulations of SSM or SSMM were then chosen based on their formation of a homogenous system and with maximum solubilization potential for paclitaxel. These optimal SSM and SSMM formulations were then tested for bioactivity. After separation of excess drug by centrifugation, mean particle size and morphology of particles in the supernatant were determined by quasi-elastic light scattering (QELS) and transmission electron microscopy (TEM) briefly described below.

Particle size distribution and mean diameter of the prepared aqueous dispersions of paclitaxel were determined by quasi-elastic light scattering using a NICOMP 380 Submicron Particle Sizer (Santa Barbara, Calif.) equipped with a 5 mW Helium-Neon laser at 632.8 nm and a temperature controlled cell holder as described previously [Alkan-Onyuksel et al., Pharm Res. 11:206-212 (1994)]. The mean hydrodynamic particle diameter, \overline{d}ₕ was obtained from the Stokes-Einstein relation using the measured diffusion of particles in solution (η=0.933, T=23° C., n=1.33). Data was analyzed in terms of volume and intensity weighted distributions. Each reported experimental result is the average of at least 3 \overline{d}ₕ values obtained from analysis of the autocorrelation function accumulated for at least 20 minutes.

The morphology of paclitaxel in the presence and absence of PEG(2000)-DSPE was visualized by transmission electron microscopy (TEM) using negative staining. A drop of the prepared paclitaxel dispersion with PEG(2000)-DSPE (molar ratio, 0.16) and without PEG(2000)-DSPE was placed on a carbon coated copper grid and stained with 1% phosphotungstic acid. After air-drying for 2-3 minutes it was then viewed under an electron microscope (JEOL 100CX) and photographed.

The solubilization potentials of SSMM and SSM for paclitaxel were determined by RP-HPLC. The clear aqueous dispersion was diluted with methanol. 20 μl of each sample was injected at least three times into a μBondapak C-18 column, 3.9 mm×30 cm (Waters, Milford, Mass.) equipped with a C18 column guard. The column was eluted with acetonitrile/water (60:40) at 1.0 ml/min (Waters 600). Detection was by UV absorption measurement at 227 nm (Waters 490). Peak areas were calculated by interfacing the detector to an electronic integrator (Hewlett Packard). The drug concentration was calculated from standard curves. The assay was linear over the tested concentration range and there was no interference of the phospholipid with the assay.

The cytotoxic activity of paclitaxel in SSMM, SSM and dimethyl sulfoxide (10% DMSO) was determined against human breast cancer cells (MCF-7; ATCC#HTB-22). The cell line was maintained in RPMI 1640 medium containing 10% fetal bovine serum and 1.0% antibiotics (penicillin and streptomycin), in a 5% carbon dioxide humidified atmosphere at 37° C. Optimum solutions of paclitaxel-SSM and paclitaxel-SSMM chosen from the solubilization studies were used as the test solutions. A 10% dimethyl sulfoxide (DMSO) solution of paclitaxel was also tested as a control. Drug free simple micelles (SSM) and mixed micelles (SSMM) in 0.01M HEPES buffer, pH 7.4 were also prepared at the same concentrations as the test solution and were used as controls. Solvents, 10% DMSO and HEPES buffer were tested at the highest concentration used in the formulations. All the samples were prepared and tested in triplicate.

The procedure used to test the in vitro cytotoxic activity of the formulation has been previously described [Likhitwitayawuid et al., J. Nat Prod. 56:30-38 (1993)]. Briefly, samples were prepared as described earlier and serial. dilutions were made to obtain paclitaxel concentrations ranging from 0.0013 to 4 μg/ml using the respective solvent that is either HEPES buffer or 10% DMSO. 190 μL of cell suspension at a density of 6×10⁴/ml were plated in a 96-well plate. 10 μl/well of the test solutions and controls were added to the microtiter plates. Control groups were also added in which 10 μl of the solvents were added. Each sample was evaluated in triplicate. The plates were then incubated for three days in a 5% CO₂ humidified atmosphere at 37° C. After the incubation period the cells were fixed to the plates by adding 100 μl well of cold 20% trichloroacetic acid (TCA) and incubating for 1 hr at 4° C. The plates were then washed, air-dried and stained with 100 μl/well of 0.4% Sulforhodamine B in 1% acetic acid for 30 min. The plates were then washed with 1% acetic acid, rinsed and 10 mM Tris buffer (200 μl/well) added. The optical density was then read at 515 nm. The optical density readings obtained for the solvent controls were used to define 100% growth after correcting for the value obtained for the zero day control. These values were then expressed as a % survival and ED₅₀ values calculated using non-linear regression analysis (percent survival versus concentration).

All the data are expressed as means±standard deviation (SD). Solubilization potential of SSMM for paclitaxel is represented as the amount of paclitaxel solubilized per ml of dispersion. The increase in solubilization with increase in total lipid amount for SSMM was determined to be linear by regression analysis and R-square value and equation to the line determined. Cytotoxic activity was expressed as percentage survival of the cells and compared to baseline using repeated measures analysis of variance with Neuman-Keuls post hoc test. ED₅₀ values were calculated for each formulation and compared statistically using one-way analysis of variance. A p-value<0.05 was considered statistically significant.

Mean hydrodynamic diameter of P-SSMM and P-SSM were 13.1±1.1 nm and 15±nm(n=3) respectively. SSMM solubilized 1.5 times more paclitaxel than SSM for the same total lipid concentration. Solubilized paclitaxel amount increased linearly with an increase in lipid concentration. A therapeutically relevant lipid concentration (15 mM) of SSMM solubilized 1321±48 μg/ml of paclitaxel. Paclitaxel in the absence of sufficient SSM, aggregated to form lipid-coated crystals. P-SSMM, P-SSM and paclitaxel in 10% DMSO had comparable cytotoxic activities against MCF-7 cells.

This in vitro study demonstrated that a lipid based drug delivery system, SSMM, is suitable for the solubilization of water insoluble drugs such as paclitaxel. SSMM solubilized higher concentrations of paclitaxel than SSM and both formulations showed significant cytotoxic activity against cultured MCF-7 cells. SSMM showed increased solubilization potential compared to SSM while retaining all its advantages, and therefore can be used as an improved lipid based carrier, for water-insoluble drugs. This study has demonstrated the potential for SSM and SSMM containing paclitaxel as effective chemotherapeutic delivery systems.

### EXAMPLE 24

According to this example, the stability of pegylated phospholipids was studied to determine whether the interaction between pegylated phospholipids, such as methoxy-PEG-distearoyl phosphatidylethanolamine (DSPE-PEG) and a model protein, such as myelopoietin (MPO), is governed by incorporation of the protein into micelles or by lipid coating of individual protein molecules. Recent studies have shown that biocompatable PEGylated phospholipids, such as DSPE-PEG 5000 interacted with and stabilized a model cytokine interleukin-2 (IL-2) (Kirchhoff et al., Proc. Controlled Release of Bioactive Materials, abstr. #5188, 2001). The impact of PEG chain length and various molar ratios on the interaction/physical complexation of PEGylated phospholipid with a chimeric cytokine, MPO, was assessed.

The physical instability of proteins leads to aggregation/precipitation, and/or conformational changes resulting in the loss of bioactivity. Previously, it had been reported that the interaction of DSPE-PEG with several amnphipathic neuropeptides (VIP, secretin, and PACAP) (Onyuksel et al., Pharm Res 16:155-160, 1999; Gandhi et al., Peptides 23:1433-1439, 2002; and Tsueshita et al., J Appl Physiol 93:1377-1383, 2002) and the protein, recombinant human IL-2, (Kirchhoffet al., Proc. Controlled Release of Bioactive Materials, abstr. #5188, 2001) resulted in increased stability. However, the mechanism of interaction of DSPE-PEG with proteins was not clear. Therefore, this study was undertaken to determine whether the interaction between DSPE-PEG and MPO is governed by incorporation of the protein into micelles or by lipid coating of individual protein molecules.

MPO has been used for the treatment of neutropenia and thrombocytopenia (Dempke et al., Anticancer Research 20:5155-5164, 2000). It is a 33 kDa protein, which acts as a chimeric cytokine and a dual agonist; it has secondary structure 2×4 α-helical bundles (McWherter et al., Biochemistry 38:4564-4571, 1999). MPO contains 3 tryptophan (Trp) residues, and it fluorescence suggests a partially exposed Trp. Its isoelectric point is 5.6 where it is electrically neutral, chemically most stable, and physically least stable.

The preparation of DSPE-PEG 2000 and 5000:MPO dispersions was completed as follows. The lipid was dissolved in pH 5.6 buffer, vortexed, and sonicated to obtain DSPE-PEG solution. MPO stock was added to DSPE-PEG solution and allowed to equilibrate for 3 hours. MPO was incubated with DSPE-PEG micelles at the isoelectric point at room temperature for various time periods. DSPE-PEG and MPO interaction fluorescence was then measured by fluorescence spectroscopy. Fluorescence intensity was measured at various wavelengths for various molar ratios of DSPE-PEG-5000:MPO. Fluorescence emission peaks were measured with various molar ratios of DSPE-PEG 2000 and 5000:MPO (excitation 295 nm; emission 305-410 nm). Fluorescence emission peak shifts (FEPS) monitored complexation. Secondary structure post complexation was assessed by circular dichroism: scanned from 260-198nm. Up to 9 nm FEPS were observed for DSPE-PEG:MPO molar ratios of ≧50:1 indicating that PEGylated lipids interacted with protein. This effect did not change with PEG chain length. Secondary structure remained intact regardless of the DSPE-PEG:MPO molar ratio suggesting the retention of protein activity.

DSPE-PEG 2000 and 5000 made complexes with MPO in a concentration dependent manner at the isoelectric point of the protein, conserved secondary structure, and improved protein stability. Similar FEPS, observed with spontaneous complexation of DSPE-PEG 2000 and DSPE-PEG 5000 with MPO, suggests that lipid monomers coat MPO molecules rather than MPO being incorporated into DSPE-PEG micelles. Therefore, DSPE-PEG can be used as a stabilizing pharmaceutical excipient for aqueous protein formulations, and this novel paradigm could be exploited for the stabilization of therapeutic proteins in aqueous solutions. Numerous modifications and variations in the invention as set forth in the above illustrative examples are expected to occur to those skilled in the art. Consequently only such limitations as appear in the appended claims should be placed on the invention.

1. A method of treating a pathology selected from the group consisting of immune disorders, inflammatory conditions, and cancer comprising the step of administering to an individual suffering from the pathology an amount of a micelle composition effective to ameliorate conditions associated with the pathology, said micelle composition prepared by a method of comprising the steps of:
a) mixing one or more lipids wherein at least one lipid component is covalently bonded to a water-soluble polymer;
b) forming sterically stabilized micelles from lipids;
c) incubating micelles from step (b) with one or more biologically active amphipathic compound(s) under conditions in which said compound(s) becomes associated with said micelles in a more biologically active conformation, wherein at least one amphipathic compound is a member of the VIP/glucagon/secretin or IL-2 family of peptides including peptide fragments and analogs.

2. The method of to claim 1, wherein preparing the micelle composition, mixing in step (a) is carried out in an organic solvent, and forming sterically stabilized micelles in step (b) is carried out in steps comprising (i) removing the organic solvent to leave a dry film, and (ii) hydrating the dry film with an aqueous solution.

3. The method of claim 2, wherein in preparing the micelle composition, the organic solvent in step (a) is removed by evaporation or lyophilization.

4. The method according to claim 1 wherein in preparing the micelle composition, mixing in step (a) is carried out in an aqueous solution.

5. A method of treating a pathology selected from the group consisting of immune disorders, inflammatory conditions, and cancer comprising the step of administering to an individual an amount of a micelle composition effective to ameliorate conditions associated with the pathology, said micelle composition prepared in a method comprising the steps of:
a) mixing one or more lipids with one or more biologically active amphipathic compounds, wherein at least one lipid component is covalently bonded to a water-soluble polymer, and wherein at least one amphipathic compound is a member of the VIP/glucagon/secretin family of peptides including peptide fragments and analogs;
b) forming sterically stabilized micelles from the mixture of step (a) under conditions in which said compound(s) becomes associated with said micelles in a more biologically active conformation.

6. The method of claim 5 wherein in preparing the micelle composition, mixing in step (a) is carried out in an organic solvent and at least one lipid is conjugated to one or more targeting compound(s), and forming micelles in step (b) is carried out in a process comprising the steps of: (i) removing the organic solvent to leave a dry film, and (ii) hydrating the dry film with an aqueous solution, said method further comprising step of: (c) incubating said micelle products under conditions wherein the targeting compound(s) associates with said micelle products in an active conformation.

7. A method of treating a pathology selected from the group consisting of immune disorders, inflammatory conditions, and cancer comprising the step of administering to an individual suffering from the pathology an amount of a sterically stabilized crystalline composition effective to ameliorate conditions associated with the pathology, said sterically stabilized crystalline composition comprising one or more biologically active compounds which are insoluble in aqueous solution, said sterically stabilized crystalline compounds prepared by a method comprising the steps of:
a) mixing the biologically active compound(s) with one or more lipids, wherein at least one of the lipids is conjugated to a water soluble polymer and at least one biologically active compound is a member of the VIP/glucagon/secretin or IL-2 family of peptides including peptide fragments and analogs; and
b) forming sterically stabilized crystalline products.

8. The method of claim 7 where in preparing the sterically stabilized crystalline compound, mixing in step (a) is carried out in an organic solvent, and forming crystalline products in step (b) is carried out in a process comprising the steps of (i) removing the organic solvent to leave a dry film; and (ii) hydrating the dry film with an aqueous solution, said method further comprising the steps of (c) contacting said crystalline products with one or more targeting compounds; and (d) incubating said crystalline products under conditions wherein the targeting compound(s) associates with said crystalline products.

9. The method of claim 7 where in preparing the sterically stabilized crystalline compound, forming in step(b) is carried out in the steps comprising (i) removing the organic solvent to leave a dry film and (ii) hydrating the dry film with an aqueous solution.

10. The method of any one of claims 1, 5, or 7 wherein said water soluble polymer is polyethylene glycol (PEG).

11. The method of any one of claims 1, 5, or 7 wherein the micelles have an average diameter of less than about 25 nm.

12. The method of any one of claims 1, 5, or 7 wherein the combination of lipids consists of distearoyl-phosphatidylethanolamine covalently bonded to PEG (PEG-DSPE).

13. The method of any one of claims 1, 5, or 7 wherein the pathology is selected from the group consisting of autism, amyotrophic lateral sclerosis, multiple sclerosis, eneuresis, Parkinson's disease, brain ischemia, stroke, cerebral palsy (CP) sleeping disorders, feeding disorders, and AIDS-associated dementias.

14. The method of any one of claims 1, 5, or 7 wherein the pathology is selected from the group consisting of Hashimoto's thyroiditis, pernicious anemia, Addison's disease, diabetes, systemic lupus erythematosus, dermatomyositis, Sjogren's syndrome, dermatomyositis, multiple sclerosis, myasthenia gravis, Reiter's syndrome, Graves disease, inflammatory bowel disease, osteoarthritis, rheumatoid arthritis, asthma, allergies, inflammatory neuropathies (Guillain Barré, inflammatory polyneuropathies), vasculitis (Wegener's granulomatosus, polyarteritis nodosa), and rare disorders such as polymyalgia rheumatica, temporal arteritis, Sjogren's syndrome, Bechet's disease, Churg-Strauss syndrome, and Takayasu's arteritis.

15. A method of preventing VIP-induced hypotension comprising the step of administering to an individual an amount of a sterically stabilized micelle or crystalline composition effective to treat a target pathology, said sterically stabilized micelle or crystalline composition prepared by any one of the methods of claims 1-9.

16. The method of any one of claims 1, 5, or 7 wherein the micelles have an average diameter of less than about 50 nm.
